(19) United States
(12) Patent Application Publication
(10) Pub. No.: US 2024/0278008 A1
(43) Pub. Date: Aug. 22, 2024

(54) AROUSAL DEVICE AND METHODS

(71) Applicant: VIRILITY MEDICAL LTD., HOD-HASHARON (IL)

(72) Inventors: Tal GOLLAN, Rama-Gan (IL); David SALTON, Hod Hasharon (IL)

(21) Appl. No.: 18/571,862

(22) PCT Filed: Jun. 23, 2022
(86) PCT No.: PCT/IL2022/050679

§ 371 Date: Dec. 19, 2023

Related U.S. Application Data
(60) Provisional application No. 63/213,817, filed on Jun. 23, 2021.

(51) Int. Cl. A61N 1/36; A61N 1/04

(52) U.S. Cl. CPC A61N 1/36007; A61N 1/0492

(57) ABSTRACT

A sexual modulation device, including: a skin patch including a body. wherein the skin patch has a surface configured to attach the skin patch to a skin surface of an anatomical region located near a genital organ: wherein the skin patch body. includes: a central portion, at least two extensions extending from the central portion. wherein the extensions include a first extension and a second extension: at least one opening between the first extension and the second extension shaped and sized to receive a genital organ: at least two electrodes, suitable to transcutaneously deliver electrical impulses. wherein each of the at least two electrodes is located at a different extension, and at opposite sides of the genital organ: an electronic circuitry and power supply configured to deliver the electrical impulses between the at least two electrodes: and wherein the central portion is soft enough to be used during sexual intercourse.

### RELATED APPLICATION/S

This application claims the benefit of priority under 35 USC § 119(e) of U.S. Provisional Patent Application No. 63/213,817 filed 23 Jun. 2021, the contents of which are incorporated herein by reference in their entirety.

### FIELD AND BACKGROUND OF THE INVENTION

The present invention, in some embodiments thereof, relates to a device and methods for sexual arousal induction and, more particularly, but not exclusively, to a device and methods for sexual arousal induction in males and females.

U.S. Pat No. 10,493,264B1 describes “a TENS system is provided having an electrode element design accommodating improved motion mechanics and maneuverability while maintaining electrical contact. An electrode having a soft wing construction is provided such that a multi-planar flexibility is provided during operation. A range of motion hinge provides and positions at a central area of a winged configuration a housing such that both a rotational movement and an angular movement may occur while maintaining continuous, direct contact with the TENS electronics within the housing. A selected die-cut pattern of a number of designs may form a stepped-in wing configuration may further contribute to a lateral flexibility of the wings” (abstract).

U.S. Pat. No. 8,805,533B2 describes “systems and methods use an external and/or implantable pulse generator system for neuromodulation stimulation to treat sexual dysfunction by the unilateral or bilateral stimulation of a target nerve A and/or a target nerve B using one or more leads and electrodes implanted in tissue in the pelvic region. The electrical stimulation waveform may be conveyed to the target nerve A electrode for a first predetermined amount of time, and conveyed to the target nerve B electrode for a second predetermined amount of time” (abstract).

U.S. Pat. No. 6,741,895B1 describes “a vaginal probe, and method of use is disclosed for stimulation of the nerves of the vagina with the purpose of testing their reaction to stimuli in the hope of defining, and treating sexual dysfunction in women. One embodiment of the invention (20) includes a shaft (22) adapted to be inserted into the genetalia of a human female. The shaft (22) includes a stop (24), presented as a thickening of the shaft (22). The probe (20) includes at least one stimulation area (26, 28) defined on a portion of the shaft (22) so that only a selected portion of the genetalia is stimulated. The shaft (22) may have sensors for measuring the reaction of the nerves to stimulation, and the stimulation areas (26, 28) may stimulate the nerves of the vagina using temperature, vibration, electricity, and/or pressure” (abstract).

European Patent Application Publication No. EP3500335A1 describes “a non-invasive device and method for treating and improving dysfunction or disorders associated with pelvic floor are disclosed. The non-invasive device comprises a generator configured to generate a current with a predetermined intensity of milliampere at a desirable frequency; at least one electrical stimulation applicator (2) comprises a probe unit (1) configured to transmit the current to a target body surface of vulvar tissues and/or pelvic floor muscle for application of an electrical stimulation; wherein the probe unit (1) comprises a negative electrode (11) and at least one positive electrode (12) and the probe unit (1) is shaped for positioning on the target body surface of the vulvar tissues and/or the pelvic floor muscle” (abstract).

Australian Patent Application Publication No. AU2018391305A1 describes “design of neuromodulation electrodes for methods to treat pelvic floor disorders, such as urinary and/or faccal 5 incontinence, by means of electrical stimulation of the left and/or right branches of the dorsal genital nerves, or pudendal nerve, using a highly flexible electrode unit to obtain stable and comfortable contact with the cutaneous tissue or mucous membrane at or near the glans 10 of the clitoris, in close proximity of the targeted nerve, for stabile neuromodulation applications” (abstract).

### SUMMARY OF THE INVENTION

Some examples of some embodiments of the invention are listed below. An embodiment may include features from more than one example and/or fewer than all features of an example:
Example 1. A sexual modulation device, comprising:

a skin patch comprising a body, wherein said skin patch has a surface configured to attach said skin patch to a skin surface of an anatomical region located near a genital organ of a subject;

wherein said skin patch body, comprises:

a central portion, at least two extensions extending from said central portion, wherein said at least two extensions comprise a first extension and a second extension;

at least one opening between said first extension and said second extension shaped and sized to receive at least part of said genital organ;

at least two electrodes, suitable to transcutaneously deliver electrical impulses, wherein each of said at least two electrodes is located at a different extension of said first extension and said second extension, and at opposite sides of said genital organ positioned within said opening;

an electronic circuitry and power supply operationally connected to the at least two electrodes;

wherein said electronic circuitry and power supply are configured to deliver said electrical impulses between said at least two electrodes; and wherein said central portion is soft enough to be used during sexual intercourse.
Example 2. A device according to example 1, wherein at least one of, said device body, said central portion or at least an edge of said central portion is flexible.
Example 3. A device according to any one of examples 1 or 2, wherein said electronic circuitry and power supply are configured to deliver said electrical impulses to a depth of at least 3 mm into the skin.
Example 4. A device according to any one of the previous examples, wherein said central portion and/or said at least two extensions have a maximal thickness of up to 7 mm.
Example 5. A device according to any one of the previous examples, wherein said central body and/or said at least two extensions have a minimal radius of tortuosity of about 2.5 mm.
Example 6. A device according to any one of the previous examples, wherein hardness of said central body and/or said at least two extensions is in a range of 25-90 shore A.
Example 7. A device according to any one of examples 1 to 6 wherein hardness of said central body and/or said at least two extensions is in a range of 15-70 shore OO.
Example 8. A device according to any one of the previous examples, wherein said at least two extensions are shaped as wings or petals.
Example 9. A device according to any one of the previous examples, wherein said electronic circuitry and power supply are configured to deliver said electrical impulses with parameter values selected to increase blood volume in at least one external sex organ.
Example 10. A device according to example 9, wherein said genital organ comprises a Clitoris of a female subject and wherein said at least one external sex organ comprise at least one of said Clitoris, a Labia Majora, a Labia Minora, and a Vestibule of said female subject.
Example 11. A device according to example 10, wherein said skin patch surface is configured to attach said skin patch to a skin surface of a Mons Pubis of said female subject at a distance of up to 5 cm from said Clitoris, and wherein said at least one opening is shaped and sized to receive at least part of said Clitoris.
Example 12. A device according to any one of examples 10 or 11, wherein said electronic circuitry and power supply are configured to deliver said electrical impulses with parameter values selected to affect at least one nerve of an autonomic nervous system innervating said Clitoris and/or Labia, or tissues associated thereof.
Example 13. A device according to example 12, wherein said tissues associated thereof comprise at least one smooth muscle and/or at least one blood vessel associated with the Clitoris and/or the Labia.
Example 14. A device according to example 13, wherein said at least one blood vessel comprises at least one dorsal clitoral artery, at least one perineal artery, at least one deep artery of the clitoris, at least one posterior labial artery or any combination thereof.
Example 15. A device according to any one of examples 12 to 14, wherein said at least one nerve is a sympathetic nerve and/or a parasympathetic nerve.
Example 16. A device according to example 15, wherein said electronic circuitry and power supply are configured to deliver said electrical impulses with a frequency in a range of 0.5 Hz-10 Hz, selected to increase activity of said at least one sympathetic nerve and/or to reduce activity of said parasympathetic nerve.
Example 17. A device according to example 15, wherein said electronic circuitry and power supply are configured to deliver said electrical impulses with a frequency in a range of 50 Hz to 200 Hz, selected to reduce activity of said at least one sympathetic nerve and/or to increase activity of said at least one sympathetic nerve.
Example 18. A device according to example 9, wherein said genital organ comprises a base of a Penis of a male subject, and wherein said external sex organ comprises said Penis.
Example 19. A device according to example 18, wherein said skin patch surface is configured to attach said skin patch to a surface of a skin region superior to a base of said Penis, at a distance of up to 5 cm from said Penis base, and wherein said opening is shaped and sized to receive at least part of said Penis base.
Example 20. A device according to example 19, wherein said electronic circuitry and power supply are configured to deliver said electrical impulses with parameter values selected to affect at least one nerve of an autonomic nervous system innervating said penis, or tissues associated with said penis.
Example 21. A device according to example 20, wherein said tissue associated with said penis comprise at least one blood vessel and/or a Corpus Cavernosum of said penis.
Example 22. A device according to example 21, wherein said at least one blood vessel comprise at least one of, an internal pudental artery, a bulbouretheral artery, a circumfex artery, and a dorsal artery.
Example 23. A device according to any one of examples 20 to 22, wherein said at least one nerve is a sympathetic nerve and/or a parasympathetic nerve.
Example 24. A device according to example 23, wherein said electronic circuitry and power supply are configured to deliver said electrical impulses with a frequency in a range of 50 Hz-200 Hz, selected to reduce activity of said at least one sympathetic nerve and/or to increase activity of said parasympathetic nerve.
Example 25. A device according to any one of the previous examples, wherein a width of said opening between said at least two extensions is in a range of 2 cm to 20 cm.
Example 26. A device according to any one of the previous examples, wherein a length of said opening between said at least two extensions is in a range of 1 cm-10 cm.
Example 27. A device according to any one of the previous examples, wherein said electronic circuitry comprises a printed circuit board connecting said power supply to said at least two electrodes.
Example 28. A device according to any one of the previous examples, wherein said skin patch 15 body comprises at least one sensor for sensing a presence of at least one additional device, and wherein said electronic circuitry is configured to deliver said electric field according to a presence of said at least one additional device.
Example 29. A device according to example 28, wherein said electronic circuitry is configured to initiate delivery of said electric field, stop the delivery of said electric field, and/or modify the delivery of the electric field according to the presence of said at least one additional device.
Example 30. A device according to any one of examples 28 or 29, wherein said at least one additional device comprises at least one additional sexual arousal device used by at least one additional subject, at least one sex toy, and/or a condom.
Example 31. A device according to any one of examples 28 to 30, wherein said at least one 25 sensor comprises a proximity sensor or a proximity detector, and wherein said at least one sensor is configured to detect a RF tag or an electronic circuitry of said at least one additional device.
Example 32. A device according to example 31, wherein said at least one sensor comprises a Radio Frequency Identification (RFID) reader, and wherein said tag comprises a RFID tag.
Example 33. A device according to any one of the previous examples, wherein said skin patch 30 body comprises a communication circuitry, configured to receive wireless signals to a remote device, and wherein said electronic circuitry is configured to deliver and/or to stop the delivery of said electrical impulses based on signals received from said remote device.
Example 34. A device according to example 33, wherein said remote device comprises a remote control, a cellular device, a mobile device, a mobile computer, and/or a wearable device.
Example 35. A device according to any one of the previous examples, wherein said skin patch body comprises a user interface operationally connected to said electronic circuitry, configured to deliver a human detectable indication to a user of the device.
Example 36. A device according to example 35, wherein said electronic circuitry signals said user interface to deliver said human detectable indication, when the device is in a standby mode, prior to stopping the electric field delivery, when the at least two electrodes are in contact with the skin surface and/or when at least one electrode of the at least two electrodes is not in contact with the skin surface.
Example 37. A device according to any one of examples 35 or 36, wherein said user interface comprises at least one vibrator configured to deliver a sensory human detectable indication to the user of the device.
Example 38. A device according to any one of the previous examples, wherein a maximal thickness of said central portion is larger than a maximal thickness of said at least two extensions.
Example 39. A device according to any one of the previous examples, comprises an adhesive interface configured to attach said skin patch surface to said skin surface.
   Example 40. A device according to example 39, wherein said adhesive interface is replaceable.
Example 41. A device according to any one of the previous examples, wherein said device is disposable.
Example 42. A device according to any one of the previous examples, wherein said skin patch surface comprises two or more electrically conducting protrusions configured to electrically interconnect said at least two electrodes to said skin surface.
Example 43. A method for sexual modulation in a female subject, comprising:
attaching a device including a skin patch, at least two electrodes an electronic circuitry and power supply, to a skin surface of an anatomical region located near a genital organ of a female subject, prior to initiating sexual activity;
activating said device to generate and deliver an electric field into said skin surface by said at least two electrodes prior to initiating said sexual activity, wherein said electric field is delivered with parameter values selected to increase sexual arousal of said female subject;
participating in sexual activity which includes penetrative sex and/or cunnilingus of said female subject while said device is attached to said skin surface and delivers said electric field, thereby increasing sexual arousal of said female subject during said sexual activity.
Example 44. A method according to example 43, wherein said attaching comprises attaching said device at a distance of up to 5 cm from a clitoris of said female subject.
Example 45. A method according to example 44, wherein said attaching comprises attaching said device to a mons pubis of said female subject.
Example 46. A method according to any one of examples 44 or 45, wherein said attaching comprises positioning each electrode of said at least two electrodes at opposite sides of said clitoris.
Example 47. A method according to any one of examples 43 to 46, comprising delivering a human detectable indication to said female subject following activation of said device and/or prior to stopping the delivery of said electric field.
Example 48. A method according to any one of examples 43 to 47, comprising communicating with at least one remote device, and wherein said activating comprises activating said sexual arousal device according to signals received from said at least one remote device.
Example 49. A method according to example 48, wherein said activating comprises, initiating the delivery of said electric field, stopping the delivery of said electric field and/or modifying said electric field according to signals received from said at least one remote device.
Example 50. A method according to any one of examples 48 or 49, wherein said at least one remote device comprises, at least one additional sexual modulation device coupled to a subject participating in said sexual activity, a sex toy, a cellular device, a mobile device, a remote control and/or a computer.
Example 51. A method according to any one of examples 43 to 50, wherein said sexual activity comprises pelvic-pelvic contact.
Example 52. A method for sexual modulation in a male subject, comprising:

attaching a device including a skin patch, at least two electrodes an electronic circuitry and power supply, to a skin surface of an abdomen superior to a base of a penis of said male subject, prior to initiating sexual activity;

activating said device to generate and deliver an electric field into said skin surface by said at least two electrodes prior to initiating sexual intercourse, wherein said electric field is delivered with parameter values selected to increase penile rigidity of a penis of said male subject.
Example 53. A method according to example 52, comprising:

participating in sexual activity with said male subject while said device is attached to said skin surface and delivers said electric field.
Example 54. A method according to example 53, wherein said sexual activity comprises pelvic-pelvic contact.
Example 55. A method according to any one of examples 52 to 54, wherein said attaching comprises attaching said device at a distance of up to 5 cm from said penis base. Example 56. A method according to any one of examples 52 to 55, wherein said attaching
comprises positioning each electrode of said at least two electrodes at opposite sides of said penis base.
Example 57. A method according to any one of examples 52 to 56, comprising delivering a human detectable indication to said male subject following activation of said device and/or prior to stopping the delivery of said electric field.
Example 58. A method according to any one of examples 52 to 57, comprising communicating with at least one remote device, and wherein said activating comprises activating said device according to signals received from said at least one remote device.
Example 59. A method according to example 58, wherein said activating comprises, initiating the delivery of said electric field, stopping the delivery of said electric field and/or modifying said electric field according to signals received from said at least one remote device.
Example 60. A method according to any one of examples 58 or 59, wherein said at least one remote device comprises, at least one additional sexual modulation device coupled to a subject participating in said sexual activity, a sex toy, a cellular device, a mobile device, a remote control and/or a computer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic illustration showing the transmission of energy to affect nerves, blood vessels and/or muscles associated with at least one sex organ of a subject, according to some exemplary embodiments of the invention;

FIG. 1B is a schematic illustration showing arteries associated with a clitoris, labia, and perineal arteries of a female subject, according to some exemplary embodiments of the invention;

FIG. 1C is a schematic illustration of nerves innervating the female vulva, according to some exemplary embodiments of the invention;

FIG. 1D is a schematic illustration showing blood vessels of a penis, according to some exemplary embodiments of the invention;

FIG. 2 is a flow chart of a process for affecting tissue associated with genital organs and/or the genital organs themselves by electric field, according to some exemplary embodiments of the invention;

FIG. 3 is a schematic illustration showing the effect of electric field on tissue associated with female genital organs, according to some exemplary embodiments of the invention;

FIG. 4A is a block diagram of an arousal device, according to some exemplary embodiments of the invention;

FIG. 4B is a flow chart of a process for using the device during sexual intercourse, according to some exemplary embodiments of the invention;

FIG. 4C is a flow chart of a process for a self-use of the device, according to some exemplary embodiments of the invention;

FIG. 5 is a flow chart of a process for operating an arousal device, according to some exemplary embodiments of the invention;

FIG. 6A is a flow chart of actions performed by an arousal device, according to some exemplary embodiments of the invention;

FIG. 6B is a flow chart of a general feedback process, according to some exemplary embodiments of the invention;

FIG. 6C is a flow chart of a detailed feedback process, according to some exemplary embodiments of the invention;

FIG. 7A is a schematic illustration showing different ways to control the activation and/or deactivation of the device, according to some exemplary embodiments of the invention;

FIG. 7B is a schematic illustration showing communication between the device and other devices, according to some exemplary embodiments of the invention;

FIGS. 8A and 8B are schematic illustrations showing interactions between two adjacent devices, according to some exemplary embodiments of the invention;

FIG. 8C is a flow chart of a process in which two or more sexual modulation devices, for example sexual arousal devices, interact, according to some exemplary embodiments of the invention;

FIGS. 9A-9G are schematic illustrations of an arousal device, for example a female or a male arousal device, according to some exemplary embodiments of the invention;

FIGS. 10A-10C are schematic illustrations showing an arousal device attached to a skin of a female subject, according to some exemplary embodiments of the invention;

FIGS. 11A-11D are schematic illustrations showing an arousal device attached to a skin of a male subject in the abdomen area, according to some exemplary embodiments of the invention; and

FIG. 12 is a schematic illustration showing an arousal device attached to the skin surface of a perineum of a male subject, according to some exemplary embodiments of the invention.

### DESCRIPTION OF SPECIFIC EMBODIMENTS OF THE INVENTION

The present invention, in some embodiments thereof, relates to a device and methods for sexual arousal induction and, more particularly, but not exclusively, to a device and methods for sexual arousal induction in males and females.

A broad aspect of some embodiments of the invention relates to delivery of an electric field, for example as electrical impulses, for sexual modulation. In some embodiments, sexual modulation comprises increasing sexual arousal, and/or increasing penile rigidity.

An aspect of some embodiments relates to delivery of an electric field, for example electrical impulses, to a genital organ and/or associated tissue during sexual activity, with a parameter value range or parameter values selected to increase blood volume in the genital organ. In some embodiments, the sexual activity comprises penetrative sex, for example vaginal or anal penetration, cunnilingus, and/or masturbation. In some embodiments, the delivered electric field increases blood flow into the genital organ. Optionally, the delivered electric field reduces or prevents leakage of blood from the genital organ. In some embodiments, the electric field is delivered with parameter values selected for sexual modulation, for example with parameter values selected to increase sexual arousal or penile rigidity. Optionally, the electric field is delivered with parameter values selected to increase sensitivity and/or responsiveness of a sex organ, for example an external sex organ to at least one stimulus, for example an external stimulus. Optionally, the external stimulus comprises mechanical and/or tactile stimulus of the external sex organ.

According to some embodiments, the electric field is delivered with a parameter value range or parameter values selected to affect nerves of the autonomic nervous system, for example sympathetic and/or parasympathetic nerves. In some embodiments, the delivered electric field increases activity of the sympathetic system and/or reduces activity of the parasympathetic system. Alternatively, the delivered electric field increases activity of the parasympathetic system and/or reduces activity of the sympathetic system.

According to some embodiments, the electric field is delivered by a device positioned on the skin surface at an abdomen, for example lower abdomen of a subject. In some embodiments, when the device is positioned at the lower abdomen, each of at least two electrodes of the device is located at a different side of the genital organ. In some embodiments, in male subjects, the genital organ comprises a Penis or a base of the Penis. In some embodiments, in female subjects, the genital organ comprises a Clitoris and/or a Labia, for example Labia majora.

According to some embodiments, the device and device embodiments described herein are configured, for example shaped and/or sized to be used by an adult male subject or by an adult female subject.

According to some embodiments, the device is shaped and sized not to interfere with movements of a user wearing of the device, and/or movements of at least one partner of the user, during sexual activity. In some embodiments, the device is shaped and sized not to interfere with pelvic-to-pelvic contact, and/or vaginal penetration, during sexual intercourse. In some embodiments, the device the device is thin. In some embodiments, the device is a low-profile device, having a maximal thickness of up to 10 mm, for example up to 9 mm, up to 8 mm, up to 7 mm, up to 5 mm or any intermediate, smaller or larger values.

According to some embodiments, a body of the device, or at least a portion of the device body which includes an electronic circuitry and/or power supply, for example at least one battery, is soft and/or flexible. Alternatively or additionally, an external surface of the device which is opposite to a skin contacting surface of the device, is smooth, curved and/or having round edges, for example to prevent tissue damage when the device is contacted by at least one partner of the subject during sexual intercourse.

In some embodiments, the electric field is delivered to a depth of at least 3 mm, for example at least 5 mm, at least 10 mm, at least 20 mm or any intermediate, smaller or larger depth from the skin surface. In some embodiments, the electric field is delivered to a distance larger than 1.5 cm, for example larger than 2 cm, larger than 3 cm, larger than 5 cm or any intermediate, smaller or larger distance into the tissue. In some embodiments, the electric field is optionally delivered with parameter values, for example frequency, intensity, duration, electric field pulse width, electric field pulse duration, selected to affect one or more nerves of the autonomic nervous system.

According to some embodiments, penetration depth of the electric field depends on multiple variables, for example anatomical position, skin cleanliness, hydration, voltage, and waveform. In some embodiments, the electric field penetrates to a depth in a range of 1-50 mm, for example 1-10 mm, 5 mm-20 mm, 10 mm-50 mm or any intermediate, smaller or larger range of values.
According to some embodiments, the one or more nerves of the autonomic nervous system affected by the delivered electric field innervate at least one genital organ of the male or female. Alternatively, or additionally, the one or more nerves of the autonomic nervous system affected by the delivered electric field innervate at least one tissue, for example a nerve, a muscle and/or a blood vessel associated with the genital organs. In some embodiments, the delivered electric field affects one or more sympathetic and/or parasympathetic nerves. Optionally, the delivered electric field deactivates one or more sympathetic nerves. Alternatively, or additionally, the delivered electric field activates one or more sympathetic nerves.
According to some embodiments, the delivered electric field optionally induces smooth muscle relaxation, for example by the deactivating or suppressing activity of one or more sympathetic nerves and/or by the activating or promoting activity of one or more parasympathetic nerves. In some embodiments, the smooth muscle relaxation optionally results with an increase in blood flow to sex organs, for example the genital organs of a subject, for example a male subject or a female subject. In some embodiments, the delivered field optionally promotes in a female subject at least one of, enlargement of the clitoris, enlargement of clitoral erection, an increase in labia engorgement, and an increase in vaginal lubrication. In some embodiments, the delivered field optionally promotes in a male subject at least one of, relaxation and engorgement of the corpus cavernosum, and an increase in erection rigidity of the penis.

According to some embodiments, the electric field is delivered by a device attached to the skin near the genital organs, for example at a distance smaller than 30 cm, for example smaller than 20 cm, smaller than 10 cm, smaller than 5 cm, smaller than 2 cm or any intermediate, shorter or longer distance from the female genital organs or the tissue associated thereof.

According to some embodiments, the device, optionally comprising at least one pulse generator, at least one electrode and a power source, is shaped and sized to be attached to the skin surface of a male or a female subject. In some embodiments, the device is shaped and sized to be attached, at least partly or completely, to a lower region of a male abdomen at a distance of up to 20 cm, for example up to 15 cm, up to 10 cm, up to 5 cm or any intermediate, shorter or longer distance from a penis. Alternatively, the device is shaped and sized to be attached, at least partly or completely, to a perineum region of a subject, for example between a posterior aspect of the scrotum and the anus. In some embodiments, the device is shaped and sized to be optionally attached, at least partly or completely, to a lower region of a female abdomen at a distance of up to 20 cm, for example up to 15 cm, up to 10 cm, up to 5 cm or any intermediate, shorter or longer distance from a clitoris, for example from the clitoral hood or the clitoral glans. In some embodiments, the device is shaped and sized to be optionally attached, at least partly or completely, to a skin surface of the mons pubis of a female subject. In some embodiments, the device, for example at least one electrode of the device, is shaped and sized to be optionally attached, at least partly to a skin surface on both sides of the vulva.

According to some embodiments, the device is shaped and sized to be optionally attached to the skin surface of a subject without obstructing the vagina and/or the anus.

According to some embodiments, the delivered electric field optionally penetrates through the skin and affects at least one of one or more nerves, one or more muscles, one or more blood vessels, associated with the female genital organs. Alternatively, or additionally, the delivered electric field affect directly and/or indirectly one or more of the genital organs. In some embodiments, the genital organs comprise external genital organs, for example the mons pubis, the labia majora, the labia minora, Bartholin glands and the clitoris, and/or the G-spot. In some embodiments, the delivered electric field penetrates through the skin into the vulva, and affects tissue and/or organs of the vulva, for example the external sex organs.

According to some embodiments, a subject, for example a male or a female subject, applies the device to the skin surface before sexual activity, for example before cunnilingus and/or sexual intercourse, for example at least 15 minutes, at least 30 minutes, at least 1 hour, at least 2 hours or any intermediate, shorter or longer time period before the sexual activity. In some embodiments, the device is activated, for example to deliver an electric field, prior to foreplay, during foreplay, prior to cunnilingus, during cunnilingus, prior to intercourse and/or during intercourse. In some embodiments, the device is activated by an input signal, for example an input triggering signal received from the user of the device or from at least one partner, for example at least one additional participant in the sexual activity. Optionally, the input triggering signal is an audio signal received by the sexual modulation device, for example a sexual arousal device or by a device paired with the sexual arousal device.

According to some embodiments, during the sexual activity, the user repeatedly activates the device. In some embodiments, after the sexual activity the device is disposed of. Alternatively, in case the device is a reusable device, the device is washed and/or recharged. Optionally, a disposable skin-electrode interface, for example a hydrogel interface and/or an adhesive patch is replaced.

According to some embodiments, at least one sexual activity partner wears a sexual arousal device. In some embodiments, the devices are in communication with each other, for example via Bluetooth or Near Field Communication (NFC) or Wi-Fi. In some embodiments, activation of at least one device is synchronized with the activation of a different device, based on the communication between the devices. In some embodiments, synchronization comprises timing of each or at least some of the electric field pulses between two or more devices.

According to some exemplary embodiments, the synchronization between the devices is performed using at least one of Bluetooth, NFC, cloud-based syncing application; using a virtual assistance technology for example Alexa, Cortana, using at least one accelerometer of the device configured to detect movement of a subject body. Optionally, during synchronization, at least one device is set as Master, while at least one different device is set as Slave (Master-Slave Synchronization). Optionally, when devices are synchronized using NFC, each pelvic thrust is identified by recurring proximity detection of the NFC sensors. In this case, intensity can increase during a thrust and decrease otherwise.

According to some embodiments, the sexual arousal device is used to treat an arousal disorder and/or erectile dysfunction. In some embodiments, the sexual arousal device is used to increase penile rigidity. Alternatively, the sexual arousal device is used by healthy subject, for example to increase arousal levels. In some embodiments, in female subjects, at least part of the device is attached to a skin surface at the mons pubis, optionally superior to the vulva. Optionally, at least part of the device, for example at least one electrode of the device is positioned along and adjacent to the labia majora. In some embodiments, in male subjects, at least part of the device is attached to the skin surface of the abdomen, optionally to a body region superior to the base of the penis. Alternatively, the device is attached at least partly to the perineum of the subject. In some embodiments, the device is attached to the perineum between a posterior aspect of a scrotum and an anus. In some embodiments, at least two electrodes of the device surround at least partly, the posterior aspect of the scrotum. Optionally, each electrode of the at least two electrodes extends towards an opposite different side of the scrotum.

According to some embodiments, the electric field is delivered with parameter values sufficient to affect at least one nerve of the autonomic nervous system, for example at least one nerve of the sympathetic system and/or at least one nerve of the parasympathetic system. In some embodiments, the electric field is delivered with intensity and/or other parameter values, sufficient to affect external genitalia, for example external sex organs, blood vessels, for example the Deep Arteries of the Clitoris, Deep Dorsal Arteries of the Clitoris, and/or anterior and/or posterior labial nerves. In some embodiments, in male subjects, the electric field is delivered with intensity and/or other parameter values sufficient to affect the internal Pudendal artery and/or the penile artery.

According to some embodiments, the delivered electric field induces electrical modulation of the autonomic (sympathetic and parasympathetic) nervous system. In some embodiments, deactivation, for example suppression of the sympathetic system, and/or activation, for example promotion of the parasympathetic system, optionally leads to smooth muscle relaxation and to an increased blood flow to sex organs. In some embodiments, increasing blood flow to sex organs promotes enlarged clitoral erection, increased labial engorgement and/or vaginal lubrication in female subjects. In some embodiments, increasing blood flow to sex organs promotes relaxation and engorgement of the corpus cavernosum, optionally leading to a rigid erection, in male subjects.

Optionally, activation, for example promotion of the sympathetic system or deactivation, for example suppression of the parasympathetic system would generally results in an opposite physiological response, of shrunken or contracted corpus cavernosum, optionally leading to detumescence.

According to some embodiments, in male subjects the delivered electric field inhibits the sympathetic system and/or stimulates the parasympathetic system, optionally leading to relaxation of one or more smooth muscles surrounding blood vessels of the corpus cavernosum. In some embodiments, relaxation of the one or more smooth muscles, increases blood flow in blood vessels delivering blood to the corpus cavernosum, optionally leading to an increased engorgement of the corpus cavernosum, and optionally a stronger, more rigid erection, longer in duration and larger in dimensions, for example an increase in length and girth of the penis.

According to some embodiments, in order to enhance a sympathetic response and/or to reduce a parasympathetic response the electric field is delivered as a symmetrical alternating square wave. In some embodiments, the electric field is delivered with an intensity in a range of 0 mA-40mA, for example 0 mA-20 mA, 10 mA-30 mA, 15 mA-40 mA or any intermediate, smaller or larger range of intensity values. In some embodiments, the electric field is delivered in a frequency range of 0.5 Hz-10 Hz, for example 0.5 Hz-5 Hz, 1 Hz-7 Hz, 3 Hz-10 Hz or any intermediate, smaller or larger range of frequencies. In some embodiments, the electric field is delivered with a pulse width in a range of 50 μs-400 μs, for example 50 μs-100 μs. 70 μs-150 μs. 100 μs-200 μs, 200 μs-400 μs or any intermediate, smaller or larger pulse width.

According to some embodiments, in order to reduce sympathetic response and/or to increase a parasympathetic response the electric field is delivered as a symmetrical alternating square wave. In some embodiments, the electric field is delivered with an intensity in a range of 0 mA-40 mA, for example 0 mA-20 mA, 10 mA-30 mA, 15 mA-40 mA or any intermediate, smaller or larger range of intensity values. In some embodiments, the electric field is delivered in a frequency range of 50 Hz-200 Hz, for example 50 Hz-70 Hz, 50 Hz-100 Hz, 50 Hz-150 Hz, 80 HZ-150 Hz or any intermediate, smaller or larger range of frequencies. In some embodiments, the electric field is delivered with a pulse width in a range of 50 μs-400 μs, for example 50 μs-100 μs, 70 μs-150 μs, 100 μs-200 μs, 200 μs-400 μs or any intermediate, smaller or larger pulse width.

According to some embodiments, the delivered electric field affects at least one muscle, for example induces contraction of the muscle. In some embodiments, the delivered electric field induces continuous contraction of the muscle. Alternatively, the electric field induces pulsatile contractions of the muscle. Optionally, inducing muscle contraction prevents leakage of blood, for example venous drainage from the genital organ.

According to some embodiments, the sexual arousal device is part of a kit, comprising a reusable sexual arousal device, replaceable one or more gel pads, and optionally an electric charger to charge the reusable device.

According to some embodiments, the one or more gel pads are disposable. In some embodiments, the one or more gel pads are selected according to a device size, for example according to a size of a surface of the device placed in contact with the skin. Alternatively or additionally, the one or more gel pads are selected according to a size, length or area of a surface of the device electrodes placed in contact with the skin. Alternatively or additionally, the one or more gel pads are selected according to an anatomical position of the body selected for attachment of the device.

According to some embodiments, the one or more gel pads are configured to be used once. In some embodiments, the one or more gel pads include a degradable material, for example a layer of degradable material comprising glue, liquid, and/or gel, configured to be degraded after a pre-determined time period, for example after at least 1 hour, after at least 1 day, or any intermediate, shorter or longer time period. Alternatively or additionally, the degradable material, for example the layer of degradable material is configured to be degraded following exposure to humidity, sweat and/or following contact with the skin. Optionally, the one or more gel pads include an indicator, for example a visual indicator indicating a degradable level of a gel pad.

According to some embodiments, the device is reusable and is configured to be attached to an add-on cover. In some embodiments, the add-on cover at least partly fits and attached to a surface of the device that remains visible after the attachment of the device to the skin. In some embodiments, the add-on cover at least partly fits and attached to a surface of the device, which is opposite to a device surface placed in contact with the skin. In some embodiments, the add-on cover includes graphics and/or text. In some embodiments, the add-on cover is configured to be reversibly attached to the device surface, for example to one or more indentations in the device surface. In some embodiments, the add-on cover is washable and/or replaceable.

According to some embodiments, a kit comprises at least one sexual intercourse-related item and the sexual arousal device. In some embodiments, the sexual-intercourse related item comprises a condom, a sex toy, an erection ring and/or a lubricant.

According to some embodiments, a kit comprises at least one drug and the sexual arousal device. In some embodiments, the at least one drug comprises cannabis and/or a drug used for the treatment of erectile dysfunction, that includes for example at least one of, sildenafil, sildenafil citrate, Tadalafil, vardenafil, and avanafil.

According to some embodiments, a kit comprises the sexual arousal device, and at least one drink or beverage containing at least one stimulant, for example caffeine, or at least one drink or beverage containing alcohol, for example an alcoholic drink, or at least one drink or beverage containing cannabis.

According to some embodiments, the sexual arousal device is in communication with a remote device, for example a cellular device or a remote computer. Optionally, the sexual arousal device is paired with the remote device. Optionally, the sexual arousal device is in communication with a software, for example a software application installed in a memory of the remote device. In some embodiments, the software is used to control the activation of the sexual arousal device, for example by modifying at least one parameter of the electric field or the delivery of the electric field. Alternatively or additionally, the software is used to order gel pads and/or other disposables associated with the sexual arousal device or with sexual intercourse, for example in a periodic supply of orders, controlled by the software. In some embodiments, the disposable associated with the sexual intercourse comprise condoms, lubricants, and/or atmosphere-generating items, for example candles.

According to some embodiments, the sexual arousal device is in communication with a remote device, for example a remote server and/or a remote cloud storage. Optionally, the sexual arousal device is in communication with the remote server and/or the cloud storage device via a cellular device or a remote computer. In some embodiments, the sexual arousal device transmits information regarding subject behavior and/or movement, to the remote server and/or cloud storage device. Additionally or optionally, the remote server and/or the cloud storage device receives clinical and/or personal data, for example from social networks, with regard to a specific subject. In some embodiments, at least one software, for example an algorithm, a database, and/or a lookup table in the remote server and/or cloud storage, analyzes the received data and information, and updates the software accordingly. Alternatively or additionally, the software is used to generate behavioral and/or clinical insights based on the received information and data. Alternatively or additionally, the software transmits at least one input signal to the sexual arousal device, based on the behavioral and/or clinical insights. In some embodiments, the transmitted at least one input signal includes an update to at least one parameter of the electric field, and/or electric field delivery protocol.

According to some embodiments, the sexual modulation device is configured to measure heart rate based caloric expenditure estimation. In some embodiments, the sexual modulation device measures heart rate based caloric expenditure estimation based on signals received from at least one sensor of the sexual modulation device. Alternatively, sexual modulation device measures heart rate based caloric expenditure estimation based on signals recorded by at least one remote sensor, for example a sensor of the remote device that is in communication with the sexual modulation device. Alternatively, the heart rate based caloric expenditure estimation is measured by a different device, and is uploaded to a software application and/or to a web-based application or social network. Optionally, the remote device and/or the sexual modulation device upload the measurements of the heart rate based caloric expenditure estimation to the software application and/or to a web-based application or social network.

In some embodiments, the heart rate based caloric expenditure estimation provides an estimation of how many calories were burned during intercourse, based on heart rate changes over a specific duration. Optionally, the heart rate based caloric expenditure estimation is measured during and/or following sexual activity, for example sexual intercourse. Optionally, the heart rate is recorded during and/or following the sexual activity.

In some embodiments, the heart rate based caloric expenditure estimation or indication thereof is presented to the user on a mobile device app and/or a web interface, and can be displayed as caloric burn per intercourse or accumulated caloric burn per specific period, or as any statistical manipulation of the heart rate based caloric expenditure estimation.

According to some embodiments, the sexual modulation device, for example the sexual arousal device, is the device described in patent U.S. Pat. No. 10,493,264B1, modified, for example to be thinner, softer and/or more flexible. Optionally or additionally, the device described in U.S. Pat. No. 10,493,264B1 is formed at least partly from a soft material, for example silicone. In some embodiments, these modifications allow using the device during sexual activity, for example during sexual intercourse with one or more partners, while preventing discomfort to a subject wearing the device, to the one or more partners participating in the sexual activity, and optionally to allow comfortable pelvic-to-pelvic contact.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Effect on Tissue

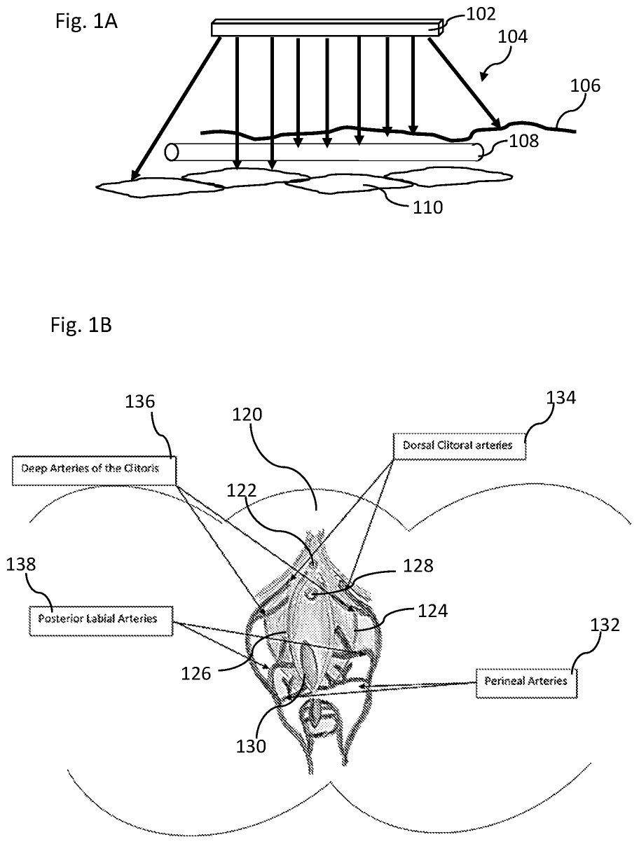

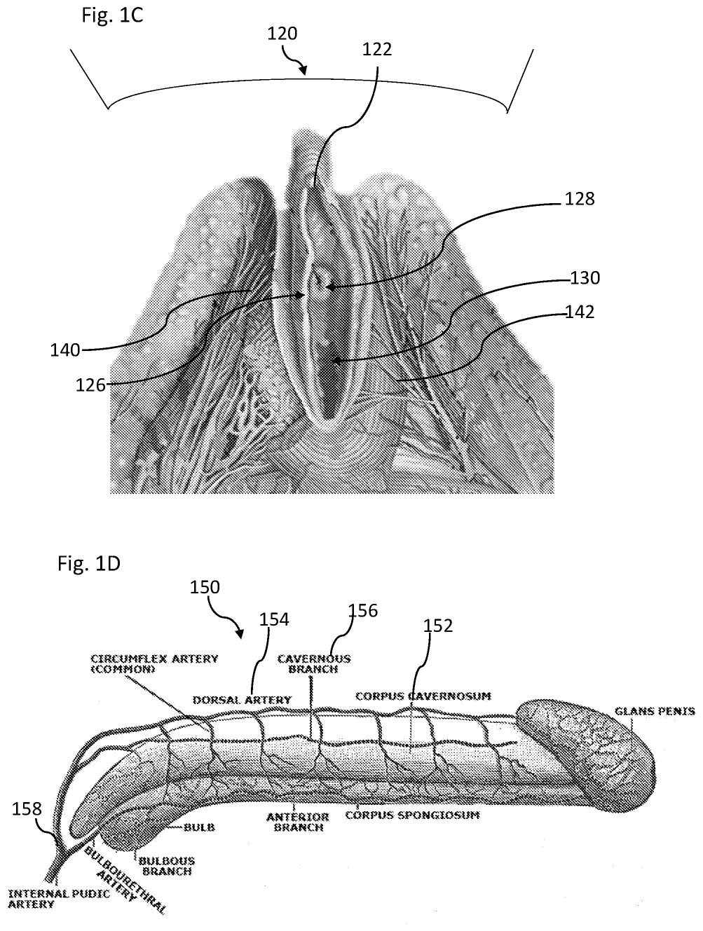

According to some exemplary embodiments, at least one energy transmitter is configured to be attached to a skin of a subject, for example near at least one genital organ. In some embodiments, the at least one energy transmitter transmits energy transcutaneously, into the skin tissue to affect one or more types of tissue associated with the at least one genital organ. Reference is now made to FIG. 1A, depicting a transmission of energy from an energy transmitter to affect tissues associated with at least one genital organ, according to some exemplary embodiments of the invention.

According to some exemplary embodiments, an energy transmitter 102, for example a transducer or an electrode is attached to a skin of a subject near at least one genital organ, for example at a distance of up to 10 cm, up to 5 cm, up to 3 cm, up to 3 mm or any intermediate smaller or larger distance from a genital organ. In some embodiments, in a male subject, the at least one genital organ comprises the penis and/or the testes. In some embodiments, in a female subject, the at least one genital organ comprises the vulva and/or the clitoris.

According to some exemplary embodiments, the transmitter, for example at least one electrode, transmits energy, for example transcutaneously, into the skin tissue. In some embodiments, the at least one electrode, for example at least two electrodes, at least 4 electrodes, at least 6 electrodes or any arrangement of electrode pairs, transcutaneously delivers an electric field into the skin tissue. In some embodiments, the energy, for example an electric field is delivered with parameter values sufficient to affect tissue in the skin.

According to some exemplary embodiments, the at least one electrode delivers an electric field with parameter values sufficient to affect at least one nerve 106. In some embodiments, the at least one nerve 106 is associated with the at least one genital organ. Optionally, neural transmission through the at least one nerve 106 controls a physiological response of the at least one genital organ, for example a physiological response related to pleasure sensation. In some embodiments, the at least one nerve 106 comprises a nerve of the autonomic nervous system, for example a nerve of the sympathetic nervous system, a nerve of the parasympathetic nervous system and/or a nerve of the enteric nervous system. In some embodiments, the delivered electric field affects neural transmission, for example at least partly blocks, increases, slows, and/or disrupts transmission through said at least one nerve 106.

According to some exemplary embodiments, the at least one electrode delivers an electric field with parameter values sufficient to affect at least one blood vessel 108, for example to affect flow within the at least one blood vessel 108. In some embodiments, the delivered electric field constricts or expands a lumen of the at least one blood vessel 108 in which blood flows. In some embodiments, the at least one blood vessel is associated with the at least one genital organ. In some embodiments, the at least one blood vessel delivers blood to, or from the at least one genital organ. In some embodiments, a delivered electric field increase flow of blood in the at least one blood vessel 108. Alternatively, a delivered electric field reduces flow of blood in the at least one blood vessel 108.

According to some exemplary embodiments, the delivered electric field affects flow in the blood vessel indirectly, for example by affecting at least one nerve, for example at least one nerve 106 optionally innervating the blood vessel.

According to some exemplary embodiments, the at least one electrode delivers an electric field with parameter values sufficient to affect at least one muscle, for example muscle 110. In some embodiments, the muscle 110 is associated with at least one genital organ. In some embodiments, the delivered electric field relaxes or constricts that muscle 110. In some embodiments, relaxation or constriction of the at least one muscle 110 modifies at least one of a shape, size, surface texture and stiffness of the at least one genital organ. In some embodiments, the delivered electric field affects relaxation or constriction levels of the muscle 110, for example by affecting at least one nerve, for example nerve 106 and/or by affecting at least one blood vessel, for example blood vessel 108.

### Exemplary Vulva Tissues

Reference is now made to FIGS. 1B and 1C depicting tissues and structures of a female subject Vulva, according to some exemplary embodiments.

According to some exemplary embodiments, at least one energy transmitter is configured to deliver an electric field, for example via at least one electrode, to affect at least one tissue associated with one or more vulva structures of a female subject. In some embodiments, the vulva of a female subject comprises a Mons pubis 120, a Clitoris 122, a Labia majora 124, a Labia Minora 126, vestibular bulbs, a vulva vestibule, Bartholin's glands, Skene's glands, a urethra 128, and a vaginal opening.

According to some exemplary embodiments, for example as shown in FIG. 1B, an energy, for example an electric field is delivered with parameter values sufficient to affect at least one blood vessel associated with one or more Vulva structures, for example Clitoral arteries, Labial arteries, and/or Perineal arteries 132. In some embodiments, the Clitoral arteries comprise, dorsal clitoral arteries 134, and deep arteries of the clitoris 136. In some embodiments, the Labial arteries comprise posterior labial arteries 138.

According to some exemplary embodiments, for example as shown in FIG. 1C, an energy, for example an electric field is delivered with parameter values sufficient to affect at least one nerve associated with Vulva structures, for example at least one nerve innervating the clitoris, at least one nerve innervating one or more blood vessels delivering blood to the clitoris, at least one nerve innervating the Labia minora and/or Labia majora, at least one nerve innervating one or more blood vessels delivering blood to the Labia majora or Labia minora. In some embodiments, the at least one nerve comprises a dorsal nerve of the clitoris 140, and/or one or more of a posterior labial nerve 142.

### Exemplary Penis Tissues

Reference is now made to FIG. 1D depicting tissues and structures of a male subject Penis, according to some exemplary embodiments.

According to some exemplary embodiments, at least one energy transmitter, is configured to deliver an electric field, for example via at least one electrode, to affect at least one tissue associated or that is part of a penis 150. In some embodiments, the energy is delivered with parameter values sufficient to allow relaxation and engorgement of a Corpus Cavernosum 152, optionally leading to rigid erection. In some embodiments, the energy, for example electric field is delivered with parameter values sufficient to affect at least one nerve associated with blood vessels delivering blood to the Penis 150, or to affect directly the blood vessels, for example to increase blood flow to the Corpus Cavernosum 152. In some embodiments, the at least one nerve comprises one or more dorsal nerves of the penis and/or cavernous nerves. In some embodiments, the at least one blood vessel comprises a dorsal artery of the penis 154, a cavernous artery 156, an internal pudendal artery 158 or branches thereof.

### Exemplary General Process for Inducing Sexual Arousal

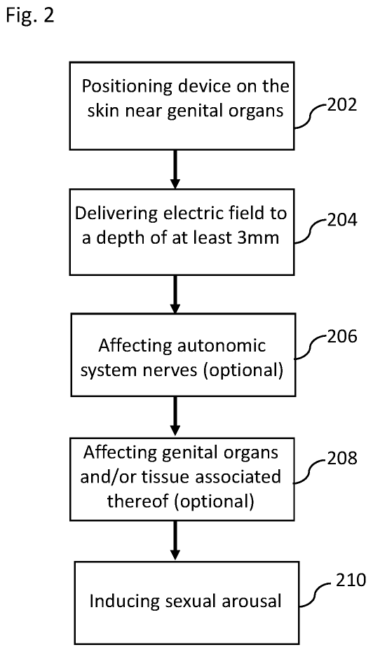

According to some exemplary embodiments, energy, for example an electric field is delivered to induce sexual arousal in a subject, for example a male or a female subject. In some embodiments, the energy is delivered in a timed relationship with sexual intercourse, for example before, during and/or after sexual intercourse. Alternatively, the energy is delivered for self-pleasuring, for example self-stimulation. Reference is now made to FIG. 2 depicting a general process for inducing sexual arousal, according to some exemplary embodiments of the invention.

According to some exemplary embodiments, a device, for example a sexual arousal device is positioned on a skin of a subject near genital organs, at block 202. In some embodiments, the device is positioned to contact at least partly the skin of the subject. In some embodiments, at least one electrode, or a skin patch of the device comprising at least one electrode is placed in contact with the subject skin at block 202. In some embodiments, the device is positioned at a distance shorter than 10 cm, shorter than 5 cm, shorter than 2 cm or any intermediate, smaller or larger distance, from a genital organ to be affected by the delivered energy.

According to some exemplary embodiments, the device is positioned at least partly at the Mons pubis of a female subject, for example at a distance smaller than 10 cm, smaller than 5 cm, smaller than 3 cm or any intermediate, smaller or larger distance from a Clitoris of the female subject. Alternatively, or additionally, the device is positioned at least partly to contact a skin surface at one or both sides of the Vulva, for example next to the Labia majora.

According to some exemplary embodiments, the device, for example at least one electrode of the device is positioned at least partly to contact a skin surface which is superior to a base of a male subject penis, for example a skin surface located between the base of the penis and the male subject abdomen. In some embodiments, the device is positioned at least partly at a distance of up to 5 cm, for example up to 3 cm, up to 1 cm or any intermediate, smaller or larger distance from a base of the penis.

According to some exemplary embodiments, an electric field is delivered, for example transcutanously delivered, to a depth larger than 3 mm into a skin tissue, at block 204. In some embodiments, the electric field is delivered to a depth larger than 3 mm, for example to a depth larger than 6 mm, larger than 1 cm, larger than 2 cm, larger than 3 cm, larger than 5 cm, or any intermediate, smaller or larger depth into the skin tissue, at block 204. Alternatively, the electric field is delivered to a depth of up to 6 cm, for example up to 5 cm, up to 3 cm, or any intermediate, smaller or larger depth into the skin tissue.

According to some exemplary embodiments, the delivered electric field optionally affects one or more nerves of the autonomic nervous system, at block 206. In some embodiments, the delivered electric field affect at least one nerve of the autonomic nervous system, for example at least one sympathetic nerve or at least one parasympathetic nerve. In some embodiments, the delivered electric field affects, for example increases neural transmission in the at least one nerve, generates neural transmission in the at least one nerve, at least partly or completely block neural transmission through the at least one nerve and/or disrupts neural transmission through the at least one nerve. Additionally, or optionally, the delivered electric field affect neural transmission between at least one nervous system structure, for example a brain, a spine and/or a supraspinal structure of the subject and a tissue, for example a blood vessel or a muscle associated with a genital organ, via the at least one nerve. Alternatively, or additionally, the delivered electric field affects neural transmission between the genital organ or the tissue associated with the genital organ, and the at least one nervous system structure of the subject via the at least one nerve.

According to some exemplary embodiments, the delivered electric field optionally affects at least one genital organ and/or tissues associated thereof, at block 208. In some embodiments, the delivered electric field affects, optionally directly, at least one muscle and/or at least one blood vessel associated with the at least one genital organ. In some embodiments, the delivered electric field affects flow, for example increases blood flow through the at least one blood vessel, for example a blood vessel supplying blood to the at least one genital organ.

According to some exemplary embodiments, the delivered electric field induces sexual arousal in the subject, at block 210. In some embodiments, the delivered electric field induces sexual arousal in the subject, at block 210. In some embodiments, the delivered electric field induces sexual arousal, for example by increasing blood flow to at least one genital organ, for example to the Clitoris of a female subject or to a Penis of a male subject. In some embodiments. the delivered electric field increases blood flow to the Corpus Cavernosum of the penis. Alternatively, or additionally, the delivered electric field induces sexual arousal in the subject by at least partly blocking draining of blood from the at least one genital organ, for example from the Clitoris or from the Penis.

Alternatively, or additionally, the delivered electric field induces sexual arousal in the subject by stimulating sensory nerves.

### Exemplary Effect on Tissue

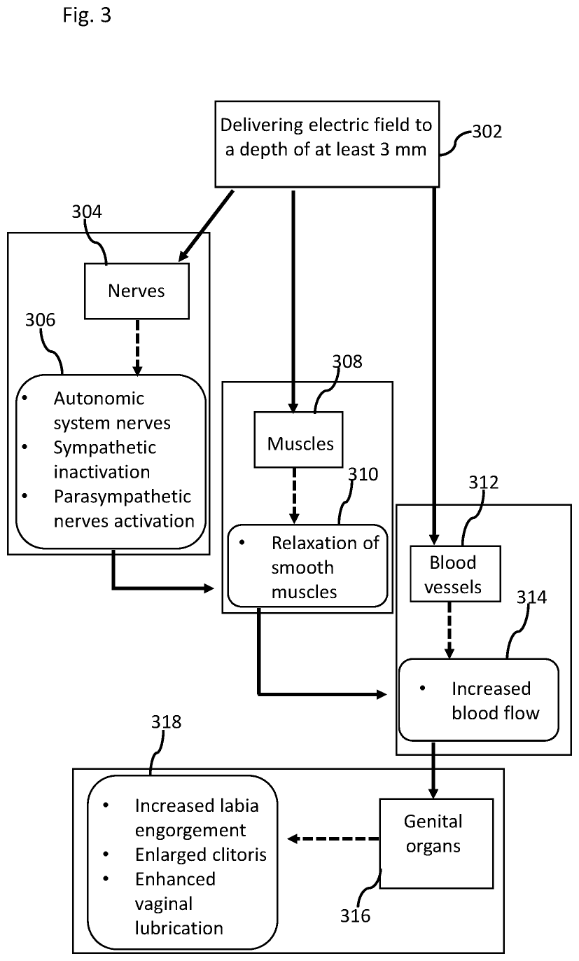

Reference is now made to FIG. 3, depicting an exemplary effect of the delivered energy on different tissue types, according to some exemplary embodiments of the invention.

According to some exemplary embodiments, an electric field is delivered to a depth of at least 3 mm, for example at least 5 mm, at least 1 cm or any intermediate, smaller or larger depth, into skin tissue, at block 302. In some embodiments, the energy is delivered, for example transcutaneously, via at least one electrode, for example 1, 2, 3, 4, 5, 6, 7, 8 electrodes placed in contact with a skin surface.

According to some exemplary embodiments, the delivered electric field affects one or more nerves at blocks 304 and 306, for example nerves associated with at least one genital organ or tissues thereof, for example with one or more genital organs of the Vulva, and/or with the Penis. In some embodiments, the one or more nerves are nerves of the autonomous nervous system, for example one or more sympathetic nerves or one or more parasympathetic nerves. In some embodiments, the electric field is delivered with parameter values sufficient to induce activation of the at least one parasympathetic nerve, for example induce neural transmission through the at least one parasympathetic nerve. Alternatively, or additionally, the electric field is delivered with parameter values sufficient to induce inactivation of at least one sympathetic nerve, for example to delay or to block neural transmission through the at least one sympathetic nerve.

According to some exemplary embodiments, the delivered electric field affects one or more muscles, at block 308. In some embodiments, the one or more muscles comprise smooth muscles and/or skeletal muscles, for example a Bulbospongiosus muscle and/or an Ischiocavernosus muscle. In some embodiments, the one or muscles are associated with one or more genital organs of the Vulva and/or with the Penis. In some embodiments, the electric field is delivered with parameter values sufficient to induce relation of at least one smooth muscle, at block 310. In some embodiments, the delivered electric field affects the one or more muscles indirectly, for example by affecting at least one nerve associated with the one or more muscles, for example smooth muscles.

According to some exemplary embodiments, the delivered electric field affects one or more blood vessels, at block 312. In some embodiments, the electric field is delivered with parameter values sufficient to increase blood flow in the blood vessels, at block 314. In some embodiments, the delivered electric field affects the one or more blood vessels indirectly, for example by affecting at least one nerve at block 304 and/or by affecting at least one muscle at block 308.

According to some exemplary embodiments, the delivered electric field affects genital organs of a subject, at block 316. In some embodiments, the delivered electric field affects genital organs of a Vulva of a female subject, for example as described at block 318. In some embodiments, the delivered electric field affects a penis of a male subject. In some embodiments, affecting a genital organ of a subject comprises modifying a shape, size, and/or a surface texture of the genital organ. In some embodiments, the delivered electric field affects the genital organs indirectly, for example by affecting at least one nerve, at least one muscle and/or at least one blood vessel.

According to some exemplary embodiments, the electric field is delivered with parameter values sufficient to increase Labia engorgement. Alternatively, or additionally, the electric field is delivered with parameter values sufficient to enlarge the Clitoris. Alternatively, or additionally, the electric field is delivered with parameter values sufficient to enhance vaginal lubrication. In some embodiments, in a male subject, the electric field is delivered with parameter values sufficient to increase penile rigidity and/or to induce erection of the penis.

### Exemplary Device

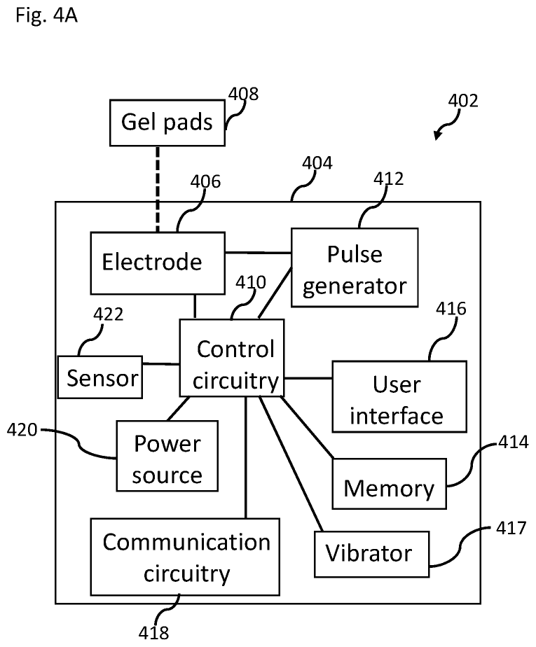

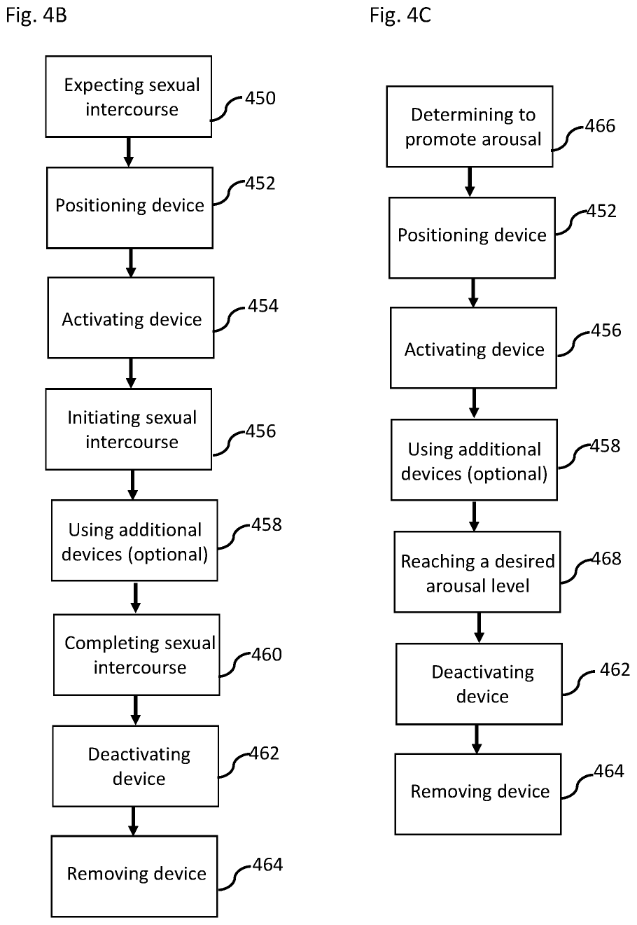

According to some exemplary embodiments, a sexual arousal inducing device is configured to be removably attachable to a skin surface of a subject, and to deliver an electric field, optionally transcutanously, with parameter values sufficient to sexually arouse the subject. Reference is now made to FIG. 4A depicting a sexual arousal device, according to some exemplary embodiments of the invention.

According to some exemplary embodiments, a device 402, for example a sexual arousal device, comprises a body 404 for example housing. In some embodiments, the device body 404 has at least one surface which is shaped and sized to be attached to a skin surface of a subject. In some embodiments, the at least one surface is smooth, for example to prevent injury to the skin surface. In some embodiments, at least a portion of the body 404 is flexible, for example to allow bending of the device 402, optionally to conform to an anatomy of the subject. Alternatively, or additionally, bending of the device 402 allows to prevent discomfort to the subject, for example during sexual intercourse. In some embodiments, that at least one flexible portion of the body has a minimal bending radius larger than 0.5 mm, 1 mm, 2 mm, 3 mm, or any intermediate, smaller or larger value.

According to some exemplary embodiments, at least a portion of the body 404 or the entire body 404 has a minimal radius of tortuosity of about 2 mm, for example a minimal radius of tortuosity of about 2.5 mm, a minimal radius of tortuosity of about 3 mm, a minimal radius of tortuosity of about 3.5 mm, a minimal radius of tortuosity of about 4 mm, or any intermediate, smaller or larger value.

According to some exemplary embodiments, at least a portion of the body 404 or the entire body 404 has a hardness in a range of 15-70 shore OO, for example 15-30 shore OO, 20-40 shore 00, 20-50 shore 00, 30-60 shore OO or any intermediate, smaller or larger range of values.

According to some exemplary embodiments, at least a portion of the body 404 or the entire body 404 has a hardness in a range of 25-90 shore A, for example 25-50 shore A, 30-70 shore A, 50-90 shore A or any intermediate, smaller or larger range of values.

According to some exemplary embodiments, the device body 404 is thin, and has a maximal thickness in a range of 1 mm-15 mm, for example in a range of 1 mm-5 mm, 1 mm-10 mm, 3 mm-8 mm, 5 mm-10 mm, 7 mm-15 mm or any intermediate, smaller or larger thickness range. In some embodiments, a thickness of the device, for example a thickness of a portion of the device containing the power source, for example power supply or battery, is up to 10 mm, for example up to 8 mm, up to 7 mm, up to 5 mm or any intermediate, smaller or larger value. In some embodiments, a weight of the device 402 is in a range of 5 grams (gr)-50 gr, for example 5 gr-15 gr, 10 gr-20 gr, 15 gr-40 gr, 15 gr-50 gr, 20 gr-40 gr or any intermediate, smaller or larger range of weights.

According to some exemplary embodiments, the device 402 comprises at least one electrode 406, for example 2, 4, 6, 8, 10 or any larger number of electrodes. In some embodiments, the at least one electrode is positioned in the surface of the device body contacting the skin, or on the device body surface. In some embodiments, the surface of the device body contacts the skin via an adhesive, for example a gel pad 408. In some embodiments, the gel pads are disposable.

According to some exemplary embodiments, the device 402 comprises a control circuitry 410 operationally connected to the at least one electrode 406. In some embodiments, the device 402 comprises at least one pulse generator, for example pulse generator 412. In some embodiments, the pulse generator 412 is operationally coupled to the at least one electrode 406. Additionally, or optionally, the pulse generator 412 is operationally connected to the control circuitry 410. In some embodiments, the control circuitry 410 signals the pulse generator 412 to generate and deliver an electric field to the at least one electrode 406.

According to some exemplary embodiments, the device 402 comprises a memory 414 for storing values of electric field parameters, for example pulse width, frequency, intensity, and/or duration. In some embodiments, the control circuitry 410 signals the pulse generator 412 to generate an electric field with parameter values stored in the memory 414.

According to some exemplary embodiments, the device 402 optionally comprises at least one user interface 416 configured to receive an input signal, for example from a user of the device. Alternatively, or additionally, the at least one user interface 416 is configured to deliver an indication signal, for example a human detectable indication signal. In some embodiments, the user interface 416 comprises at least one button and/or switch, for example to receive the input signal. Alternatively, or additionally, the user interface 416 comprises at least one audio and/or visual generator, for example to generate the indication signal. Alternatively, or additionally, the user interface 416 comprises at least one vibrator, for example to generate an indication signal by vibration.

According to some exemplary embodiments, the device 402 comprises at least one vibrator 417, for example at least one vibration motor. In some embodiments, the at least one vibrator 417 is operationally coupled to the control circuitry 410. In some embodiments, the control circuitry 410 is configured to activate the vibrator 417 in a timed relation, for example before, during and/or after, to the electric field delivery, for example to enhance an arousal of the subject. Optionally, the control circuitry 410 is configured to activate the vibrator 417 in synchronization with the activation of the pulse generator 412 generating the electric field.

According to some exemplary embodiments, the control circuitry 410 is configured to activate the at least one vibrator 417 in order to deliver an indication to the subject by tactile sensation. In some embodiments, the control circuitry 410 activates the at least one vibrator 417 to indicate, for example, that the device is positioned at a selected target area and/or in a desired orientation. Alternatively, the control circuitry 410 activates the at least one vibrator 417 to indicate, for example, that the device is not positioned at a selected target area and/or is not positioned in a desired orientation. Optionally, the control circuitry 410 activates the at least one vibrator to provide any indication with regard to the device operation, for example as described with respect to the user interface 416. Optionally, the control circuitry 410 activates the at least one vibrator 417 to provide indications to a user during sexual intercourse, for example not to interfere with the user comfort during sexual intercourse.

According to some exemplary embodiments, the user interface 416 is used to receive an input signal, for example to move the device activation between two activation states, for example between an inactive state to a standby state, between a standby state and an active state, between an active state and a standby state, and/or between a standby state and an inactive state. In some embodiments, the user interface 416 is operationally, for example electrically connected, to the control circuitry 410. In some embodiments, the control circuitry 410 signals the user interface 416 to generate an indication. In some embodiments, the control circuitry, receives an input signal from the user interface 416.

According to some exemplary embodiments, the device 402 optionally comprises at least one communication circuitry, for example communication circuitry 418, operationally connected to the control circuitry 410. In some embodiments, the communication circuitry is configured to transmit and/or receive a signal, for example a wireless signal, from an additional device. In some embodiments, the additional device comprises a wearable sensor, a wearable device, a watch, a remote control, a mobile device, a cellular device, a computer, a remote server, a cloud storage, and/or a virtual assistant device. In some embodiments, the control circuitry 410 signals the pulse generator 412 to generate and deliver an electric field via the at least one electrode 406, based on a signal received by the communication circuitry 418 from the additional device.

According to some exemplary embodiments, the wireless signal comprises at least one of radiofrequency signal, a Wi-Fi signal, a Bluetooth signal, and/or an infra-red signal.

According to some exemplary embodiments, the device 402 comprises at least one power source 420, operationally connected to the control circuitry 410. In some embodiments, the power source 420 comprises at least one battery, for example 2, 3, 4 or any larger number of batteries. In some embodiments, the at least one battery is a replaceable battery. Alternatively or additionally, the at least one battery is a rechargeable battery. Alternatively, the power source comprises a capacitor.

According to some exemplary embodiments, the at least one power source 420 stores electrical power which is sufficient for activating the device 402 for a time period of up to 5 minutes, up to 10 minutes, up to 30 minutes, up to 60 minutes, or any intermediate, smaller or larger duration.

According to some exemplary embodiments, the device 402 optionally comprises at least one sensor, for example sensor 422. In some embodiments, the at least one sensor 422 is configured to measure at least one physiological parameter indicating an activation level of the sympathetic nervous system or the parasympathetic nervous system. In some embodiments, the at least one sensor 422 is configured to measure at least one of temperature, blood flow, blood pressure, heart beat, electrical conductance of the skin and humidity.

According to some exemplary embodiments, the control circuitry 410 signals the pulse generator 412 to generate an electric field or to stop the generation of the electric field based on signals received from the at least one sensor. Alternatively or additionally, the device 402 is wirelessly connected to a remote sensor, for example a wearable sensor or a sensor in a vicinity of the subject, for example an optic sensor and/or a sound sensor. In some embodiments, the control circuitry 410 signals the pulse generator 412 to generate an electric field or to stop the generation of the electric field based on signals received from the remote sensor.

According to some exemplary embodiments, the control circuitry 410 is configured to deliver an electric field in response to an input signal from a user or from a different subject, for example via an input signal transmitted to the sexual arousal device from a remote device, for example a cellular device or a virtual assistance device. Alternatively or additionally, the control circuitry 410 is configured to deliver an electric field in synchronization with an additional device, for example an additional sexual arousal device. Alternatively or additionally, the control circuitry 410 is configured to deliver an electric field, for example automatically, when sensing at least one of, an increase in sympathetic activity, an increase in parasympathetic activity and/or an increase in genital arousal. Optionally, the control circuitry 410 is configured to deliver an electric field based on measured values of at least one physiological parameter indicating an increase in sympathetic activity, an increase in parasympathetic activity and/or an increase in genital arousal. In some embodiments, the at least one physiological parameter comprise blood flow, temperature, heart rate, skin conductance, and/or muscle contraction.

According to some exemplary embodiments, the body 404 comprises an electrical insulation, for isolating the body 404 and/or electrical components, circuits and/or circuit boards within the body from external fluids in the vicinity of the device. In some embodiments, the fluids comprise saliva, semen and/or sweat. Alternatively or additionally, the electrical insulation is configured to prevent electrification of the user or at least one partner participating in the sexual activity, for example the sexual intercourse when contacting the device 402.

According to some exemplary embodiments, the device 402, for example the body 404, is scented. In some embodiments, the device 402 is configured to release a predetermined scent, optionally during device activation and/or during sexual intercourse. In some embodiments, the device body is coated with a scented coating. Alternatively, the device body 404 is coated with a scent absorbing coating or layer, configured to absorb a fluid that includes a selected scent.

Optionally, a kit containing the device 402 also includes a scent source configured to be coupled to the device 402, for example to the body 404. Alternatively, the scent source comprises a fluid that is sprayed onto the device body 404, for example onto the scent absorbing coating or the scent absorbing layer. In some embodiments, the scented device masks unwanted scents, for example a scent of a rubber.

### Exemplary Processes for Using the Device by a Subject

Reference is now made to FIG. 4B, depicting a process for using a device to promote sexual arousal as part of sexual intercourse, according to some exemplary embodiments of the invention.

According to some exemplary embodiments, the device is activated as part of a training session. Optionally, the device is used during sexual surrogacy. Optionally, the training session, for example the sexual surrogacy is used to determine initial parameters for the device activation, for example initial parameters for the electric field and/or electric field delivery. Optionally, the initial parameters for the electric field and/or electric field delivery, are updated during sexual intercourse and/or based on information received during sexual intercourse.

According to some exemplary embodiments, a subject expects sexual intercourse, at block 450. In some embodiments, the subject expects to begin sexual intercourse in about 60 minutes, for example about 40 minutes, about 30 minutes, about 15 minutes or any intermediate, smaller or larger time period.

According to some exemplary embodiments, the subject positions the device at a target location, at block 452. In some embodiments, the device is positioned at the perineum region, for example attached to a skin surface of the perineum, optionally between a posterior aspect of the scrotum and an anus of the subject. Alternatively, the device is positioned in close proximity to a base of a penis, for example at a minimal distance of up to 10 cm, up to 7 cm, up to 5 cm, up to 3 cm, up to 1 cm or any intermediate, smaller or larger minimal distance from the base of the penis. In some embodiments, the device is attached to a skin surface which is superior to the base of the penis. Optionally, when the device is attached to the skin surface of a male subject in a location superior to a base of the penis, each electrode of at least two electrodes of the device is located on a different, for example opposite, side of the penis base.

According to some exemplary embodiments, the device is positioned at a location close to the clitoris, for example at a minimal distance of up to 15 cm, up to 10 cm, up to 5 cm, up to 3 cm, or any intermediate, smaller or larger minimal distance from the clitoris. In some embodiments, at least a portion of the device is attached to a skin surface which is superior to the clitoris, for example to the Mons pubis of a female subject. In some embodiments, attaching at least part of the device to a skin surface at the Mons pubis positions each electrode of at least two electrodes of the device at a different opposite side of the clitoris. Alternatively, the device is attached at least partly to a perineum of a female subject, for example between a vagina and an anus of the female subject.

Optionally, after attachment of the device to the subject, for example to the skin, the device is activated in a standby state, for example according to skin impedance.

According to some exemplary embodiments, the subject activates the device to generate an electric field at block 454, for example after initiating sexual intercourse. In some embodiments, the subject activates the device prior to initiating the sexual intercourse at block 456. In some embodiments, the subject activates the device prior to penetration, for example during foreplay. Alternatively, the device is activated by at least one partner of the subject, for example by at least one additional person participating with the subject in sexual activity.

According to some exemplary embodiments, the device is activated using a user interface of the device, for example user interface 416 shown in FIG. 4A. In some embodiments, the device is activated by at least one activation button or at least one switch of the user interface 416.

Alternatively or additionally, the device is activated by transmitting a signal, for example a wireless signal to the device. In some embodiments, the wireless signal is transmitted from at least one remote device, for example a wearable device, a cellular device, a remote computer and/or a virtual assistant device.

According to some exemplary embodiments, the subject initiates sexual intercourse at block 456.

According to some exemplary embodiments, at least one additional device is optionally used, at block 458. In some embodiments, the additional device is attached to a different anatomical region on the subject body, for example the additional device is attached to the perineum of the subject. Alternatively or additionally, the at least one additional device is a device used by the subject during the sexual intercourse, for example a vibrator. Alternatively or additionally, the at least one device is a device used by at least one sexual intercourse partner, for example an arousal device attached to the skin surface of the partner. In some embodiments, the device and the at least one additional device are in communication with each other, for example are in synchronization. According to some exemplary embodiments, the sexual intercourse is completed at block 460.

According to some exemplary embodiments, the subject deactivates the device at block 462. In some embodiments, the device is deactivated after completing the sexual intercourse. In some embodiments, deactivation of the device comprises moving from an active state to a standby state. Alternatively, deactivation of the device comprises moving from an activate state to a closed state. As used herein, a closed state is a state of the device in which no power is delivered from the power supply of the device. A standby state is an activation state of the device where power is delivered from the power supply without delivery of an electric field. An active state of the device is an activation state where power is delivered from the power supply of the device, and an electric field is transmitted by the at least two electrodes of the device.

According to some exemplary embodiments, the subject removes the device, at block 464. In some embodiments, the subject detaches the device from the skin surface at block 464. In some embodiments, the device is disposable, and is disposed by the subject after a single use. Alternatively, the device is a reusable device. In some embodiments, the subject charges the power supply, for example a battery of the device after device removal.

According to some exemplary embodiments, for example as described in FIG. 4C, the device is used by the subject for self-pleasuring, for example self-stimulation.

According to some exemplary embodiments, the subject determines to promote arousal at block 466. In some embodiments, the device is positioned at block 452 and is activated at block 454. Optionally, at least one additional device is used at block 458, for example a vibrator.

According to some exemplary embodiments, the subject determines that a desired arousal level has been reached at block 468, and deactivates the device at block 462.

According to some exemplary embodiments, activation of the sexual arousal device is synchronized with an audio and/or a visual signal, for example an audio and/or visual signal received from an entertainment device, for example a mobile device, a display screen, or a cellular device. In some embodiments, an intensity, duration and/or initiation time of the delivered electric field is synchronized with a presentation or a scenario presented using the entertainment device, for example a movie, a game, a video clip, and/or an interactive scenario.

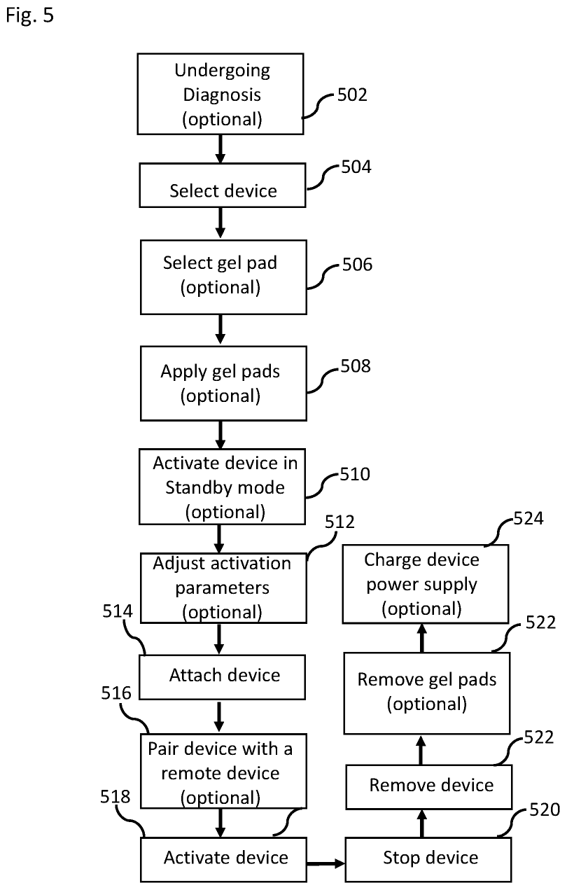

Reference is now made to FIG. 5, depicting personalized adjustment of a device for a specific subject, according to some exemplary embodiments of the invention.

According to some exemplary embodiments, a subject, for example a male subject or a female subject, optionally undergoes diagnosis at block 502. In some embodiments, the subject is diagnosed with sexual dysfunction, for example female sexual arousal disorder, male erectile disorder, female sexual interest disorder, Female Sexual Interest/Arousal Disorder (FSIAD). In some embodiments, during the diagnosis, a target region for attaching the device to the subject is selected. Additionally, during diagnosis at least one parameter of the target region is determined, for example anatomy, shape, size, thickness of fat layer, texture and/or smoothness of a skin surface at the target region

According to some exemplary embodiments, a device is selected at block 504. In some embodiments, the device is selected according to the diagnosed disease or disorder. In some embodiments, the device is selected according to the at least one parameter determined during diagnosis at block 502. In some embodiments, the device is selected to fit a shape, size, and/or thickness of fat layer at the selected target region. In some embodiments, the device is selected according to a predetermined distance between a target region for the attachment of the device and at least one tissue target. Alternatively or additionally, a device is selected according to a maximal and/or minimal distance between at least two electrodes of the device that is required in order to achieve a desired effect on a target tissue, for example on at least one nerve, at least one muscle and/or at least one blood vessel associated with a genital organ.

According to some exemplary embodiments, optionally, gel pads are selected at block 506. In some embodiments, the gel pads are selected according to the at least one detected parameter. In some embodiments, the gel pads are selected according to the texture and/or smoothness of a skin surface at the target region. In some embodiments, the gel pads are selected according to a subject anatomy, for example as determined at block 502.

According to some exemplary embodiments, the gel pad is optionally applied at block 508. In some embodiments, a gel pad selected at block 506 is applied. In some embodiments, the gel pad is applied prior to initiation of sexual intercourse or prior to self-stimulating.

According to some exemplary embodiments, the device is optionally activated in a standby state, at block 510. In some embodiments, the device is optionally activated in a standby state before attaching the device to the body. In some embodiments, the device is activated in a standby state, for example by removing an electric isolator from a power supply, for example a battery of the device. Alternatively, the device is activated in a standby state by delivering an input signal to the device, for example via the communication circuitry 418 and/or using the user interface 416 shown in FIG. 4A.

According to some exemplary embodiments, at least one activation parameter is adjusted at block 512. In some embodiments, the at least one activation parameter comprises duration of electric field delivery, intensity, amplitude, frequency, pulse width, number of pulses per a predetermined time period, and/or interphase interval (IPI). In some embodiments, a distance between at least two spaced-apart electrodes of the device is optionally adjusted at block 512. In some embodiments, the distance between the at least two electrodes is adjusted according to the anatomy of the subject and/or according to a maximal and/or minimal distance between the at least two electrodes of the device, that is required in order to achieve a desired effect on the target tissue.

According to some exemplary embodiments, the at least one activation parameter is adjusted, for example by delivering an input signal to the user interface of the device. Alternatively, the at least one parameter is adjusted using an application program or a software installed on a remote device, for example a mobile device, a computer, and a cellular device, optionally in wireless or wires communication with the device, via the communication circuitry of the device.

According to some exemplary embodiments, the device is attached to the skin of the subject at block 514. In some embodiments, the device is attached to the body at a predetermined target region, for example to a perineum of a male or a female subject. Alternatively, the device is attached at least partly to a skin surface of the Mons Pubis of a female subject, or to a skin surface superior to a base of a penis of a male subject. In some embodiments, the at least one activation parameter is optionally adjusted, for example as described at block 512, after attaching the device to the body.

According to some exemplary embodiments, following attachment of the device to the body, the device is optionally paired to a remote device, at block 516. In some embodiments, the device is optionally paired to the remote device prior to the attachment to the body. In some embodiments, the pairing comprises establishing a wireless communication channel between the device, for example the arousal device and the at least one remote device, via wireless signals.

According to some exemplary embodiments, the device is activated at block 518, for example to generate and deliver the electric field. In some embodiments, the device is activated in response to an input signal received from a subject onto which the device is attached, or any other subject, for example an additional participant in the sexual intercourse. Alternatively or additionally, the device is activated in response to a wireless signal received from a remote device via the communication circuitry. Alternatively or additionally, the device is activated in response to signals received from at least one sensor of the device, for example sensor 422 and/or a sensor in communication with the device.

According to some exemplary embodiments, the delivery of the electric field is stopped at block 520. In some embodiments, the delivery of the electric field is stopped, for example in response to a signal received by the user interface and/or in response to a signal received from a remote device. Alternatively or additionally, the delivery of the electric field is stopped, for example in response to a signal received from at least one sensor of the device, or at least one sensor in communication with the device.

According to some exemplary embodiments, the device is removed, for example detached, from the subject body at block 522. In some embodiments, the device is detached from the skin surface of the subject at block 522. In some embodiments, at least part of the device is optionally detached from the skin surface, for example a power supply and/or a control circuitry, and at least part of the device, for example the at least two electrodes optionally remain attached to the body. Optionally, the device is disposable. Optionally, the device is a single use device.

According to some exemplary embodiments, the at least one gel pad is optionally removed from the body of the subject, at block 522. In some embodiments, the at least one gel pad is disposable.

According to some exemplary embodiments, a power supply of the device, for example at least one battery, is optionally charged at block 524. Alternatively, the power supply of the device is replaced.

### Exemplary Device Activities

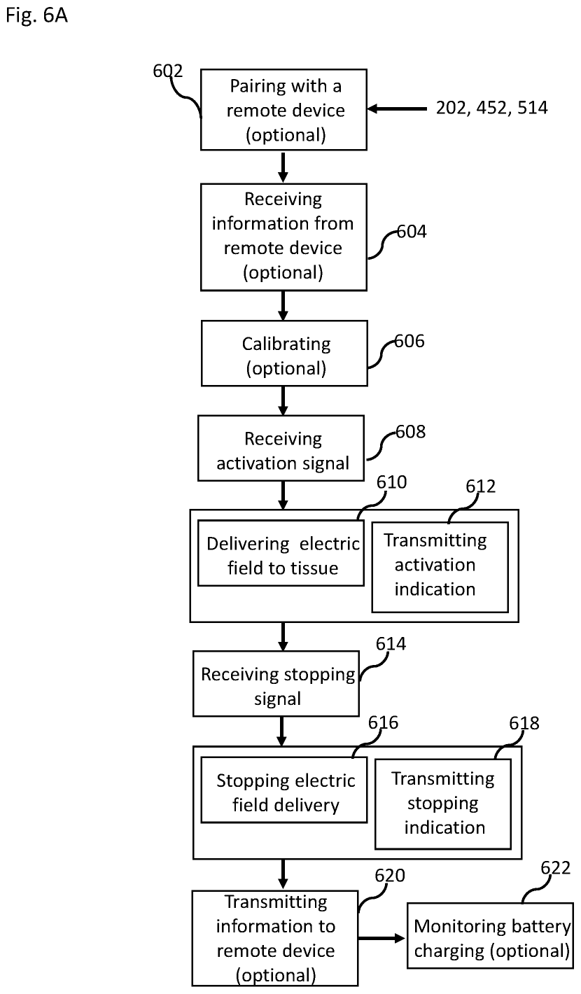

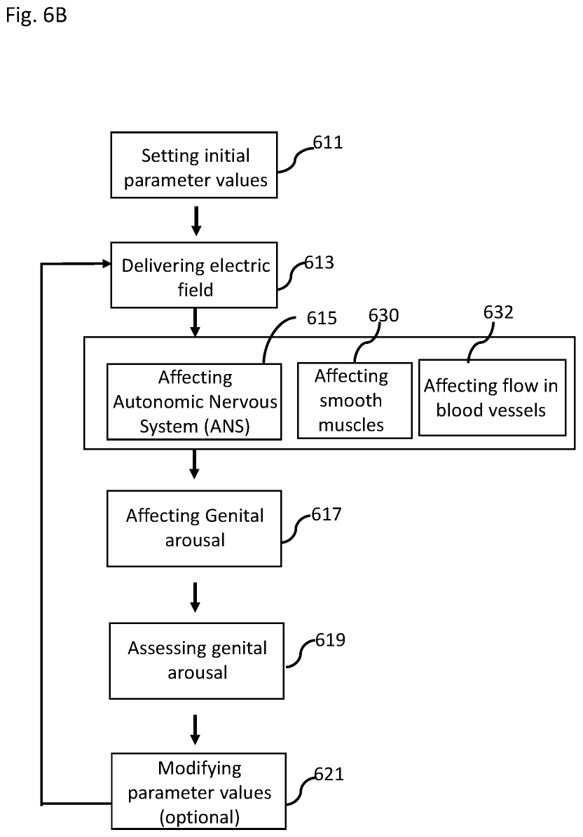

Reference is now made to FIG. 6A, depicting a flow chart of a process performed by the sexual arousal device, according to some exemplary embodiments of the invention.

According to some exemplary embodiments, following attachment of the sexual arousal device to the body of the subject, the sexual arousal device is paired with at least one remote device, at block 602. In some embodiments, the pairing comprises generating a communication channel between the sexual arousal device and the remote device. In some embodiments, the communication channel is a wireless communication channel, allowing the sexual arousal device to receive signals from the remote device and/or to transmit signals to the remote device. According to some exemplary embodiments, the sexual arousal device optionally calibrates at least one parameter of the electric field, at block 606. In some embodiments, the sexual arousal device calibrates at least one parameter of the electric field, for example to adjust the at least one parameter to a specific subject. In some embodiments, the at least one calibrated parameter comprises minimal and/or maximal intensity of the electric field, frequency, pulse width, number of pulses per time period. In some embodiments, the sexual arousal device optionally performs calibration according to at least one calibration program stored in a memory of the sexual arousal device.

According to some exemplary embodiments, the sexual arousal device receives an activation signal, at block 608. In some embodiments, the sexual arousal device receives an activation signal via a user interface, for example the user interface 416 shown in FIG. 4A, or via a communication circuitry, for example the communication circuitry 418. In some embodiments, the activation signal is received from a user of the device, or a sexual intercourse partner. In some embodiments, the activation signal is received from a remote device, for example from a mobile device, a cellular device, a remote computer and/or a virtual assistant device.

According to some exemplary embodiments, the activation signal is received from at least one sensor of the sexual arousal device, for example the sensor 422. In some embodiments, the activation signal is received from the sensor, for example when a sexual intercourse partner is detected or is at a predetermined proximity to the sexual arousal device. Alternatively or additionally, the activation signal is received from the at least one sensor, for example when the sexual arousal device is in a predetermined proximity to an additional sexual arousal device, or in proximity to a tag, for example a RFID tag or Near Field Communication (NFC).

According to some exemplary embodiments, the sexual arousal device generates and delivers the electric field at block 610. In some embodiments, the sexual arousal device delivers the electric field according to at least one activation protocol or electric field parameters stored in the memory of the device, for example the memory 414 shown in FIG. 4A. In some embodiments, the sexual arousal device delivers the electric field as previously described at blocks 204 shown in FIG. 2, and/or at block 302 shown in FIG. 3.

According to some exemplary embodiments, the sexual arousal device transmits an activation indication, at block 612. In some embodiments, the sexual arousal device transmits the activation indication when initiating the delivery of the electric field or during the delivery of the electric field. In some embodiments, the activation is a human detectable indication, for example a tactile indication optionally delivered by vibration to the body of a subject wearing the sexual arousal device. Alternatively or additionally, the activation indication is transmitted to a remote device or is stored in a memory of the sexual arousal device.

According to some exemplary embodiments, the sexual arousal device receives a stopping signal at block 614. In some embodiments, the sexual arousal device receives the stopping signal to stop the delivery of the electric field. In some embodiments, the sexual arousal device receives the stopping signal from the user interface, from a remote device via the communication circuitry and/or from the at least one sensor, for example when the sexual arousal device is not at a desired proximity to the sexual intercourse partner or to an additional device.

According to some exemplary embodiments, the sexual arousal device stops the delivery of the electric field at block 616. In some embodiments, the sexual arousal device stops the delivery of the electric field in response to the stopping signal received at block 614.

According to some exemplary embodiments, a stopping indication is transmitted at block 618. In some embodiments, the stopping indication is transmitted to the subject wearing the sexual arousal device, for example via vibration. Alternatively or additionally, the stopping signal is transmitted to the remote device. In some embodiments, the stopping indication is delivered at least 1 second, for example at least 5 seconds, at least 10 seconds, at least 30 seconds or any or any intermediate, shorter or longer time period before stopping the delivery of the electric field, for example to prepare the subject to a physiological change due to the stopping of the electric field delivery.

According to some exemplary embodiments, information regarding the activity of the sexual arousal device is optionally transmitted to a remote device, at block 620. In some embodiments, log files are transmitted to the remote device. In some embodiments, the information transmitted to the remote device is optionally used to create and/or update a database. Alternatively or additionally, the information transmitted to the remote device is optionally used to monitor the activity of the subject.

According to some exemplary embodiments, the device optionally monitors a charging of a power supply, for example at least one battery of the device, at block 622.

### Exemplary Feedback

According to some exemplary embodiments, an effect of the delivery of the electric field on activity of one or more nerves, one or more muscles and/or blood flow in blood vessels is determined. Alternatively or additionally, an activation state of the Autonomic Nervous System is determined before, during and/or after the delivery of the electric field. In some embodiments, at least one parameter of the electric field is adjusted according to the determined effect and/or according to the determined activation state.

According to some exemplary embodiments, changes in blood flow during and/or following the delivery of the electric field are measured. In some embodiments, blood volume or changes thereof are measured for example using Photoplethysmography (PPG). PPG is an optical technique used to measure changes in blood volume. In some embodiments, blood volume and/or blood flow is measured in the vagina, for example to determine Vaginal Pulse Amplitude (VPA). Alternatively or additionally, blood volume and/or blood flow is measured next to the clitoris or labia majora. for example to determine Labial Pulse Amplitude (LPA). Alternatively or additionally, blood volume and/or blood flow in the clitoris is measured, for example to determine Clitoral Blood Volume (CBV). In some embodiments, the measured blood flow, and/or blood volume or changes thereof are used as an index for female genital arousal state and response. Alternatively or additionally, the measured blood flow, and/or blood volume or changes thereof are used to generate an index for female genital arousal response, for example based on measurements taken from a plurality of female subjects. Alternatively or additionally, the measured blood flow, and/or blood volume or changes thereof are used to generate an index or are used as an index for activity of the autonomous nervous system, the sympathetic system and/or the para-sympathetic system.

According to some exemplary embodiments, blood flow, blood volume or changes thereof is monitored or measured by at least one sensor of the sexual arousal device. Alternatively or additionally, the blood flow, blood volume or changes thereof is monitored or measured by at least one sensor in communication with the sexual arousal device.

According to some exemplary embodiments, Skin Conductance Level (SCL) is measured. SCL is an electro-dermal measure caused by the activity of sweat glands that are innervated solely by the sympathetic branch (SNS) of the autonomic nervous system (ANS). In some embodiments, SCL measurements are used to monitor activity of one or more nerves of the sympathetic system. In some embodiments, SCL is monitored or measured by at least one sensor of the sexual arousal device. Alternatively or additionally, SCL is monitored or measured by at least one sensor in communication with the sexual arousal device.

According to some exemplary embodiments, Heart Rate Variability (HRV) is measured. HRV is an index marker for sympathetic activity. In some embodiments, HRV lower than baseline (in time domain) indicates a more dominant sympathetic activity. In some embodiments, HRV which is higher than baseline (in time domain) indicates a more dominant parasympathetic activity. In some embodiments, HRV analysis in frequency domain generates a sympathovagal balance, LF:HF ratio (Low Frequency: 0.04-0.15 Hz, represent the sympathetic component; High

Frequency 0.15-0.4 Hz, represent the parasympathetic component). In some embodiments, HRV measurements are derived from direct ECG measurements and/or from analyzing PPG measurements. In some embodiments, HRV measurements are derived from measurements performed by at least one sensor of the sexual arousal device and/or from measurements performed by at least one sensor in communication with the sexual arousal device.

According to some exemplary embodiments, women diagnosed with sexual arousal disorder, have overactive, or hypoactive sympathetic response. In some embodiments, delivery of the electric field modulates the activity levels of the ANS, for example, to activity levels is within a target range for a healthy sexual response.

Optionally, a healthy sexual response is predetermined per a clinical state of the female subject, age, and/or drug regime.

Reference is now made to FIG. 6B depicting a general feedback process, according to some exemplary embodiments of the invention.

According to some exemplary embodiments, initial parameter values are set, at block 611. In some embodiments, initial values of at least one parameter, related to the electric field or to the electric field delivery process, for example electric field intensity, electric field frequency, time for each pulse, pulse width, number of pulses per a train of pulses and/or interval between pulses, is set at block 611. In some embodiments, the initial values of the at least one parameter are stored in a memory of the sexual arousal device.

According to some exemplary embodiments, an electric field is delivered, at block 613. In some embodiments, the electric field is delivered according to the stored initial parameter values.

According to some exemplary embodiments, the delivered electric field affects at least one nerve of the ANS, at block 615. In some embodiments, the delivered electric field affects at least one nerve of the sympathetic nervous system and/or at least one nerve of the parasympathetic nervous system.

In some embodiments, the delivered electric field affects at least one smooth muscle, at block 630. In some embodiments, the at least one smooth muscle is affected directly by the electric field delivery. Alternatively or additionally, affecting the at least one nerve of the ANS leads to an indirect effect on the at least one smooth muscle at block 630, for example as described in FIG. 3.

In some embodiments, the delivered electric field affects flow of blood in one or more blood vessels, at block 632. In some embodiments, the blood flow is affected directly by the delivered electric field. Alternatively or additionally, affecting the at least one nerve of the ANS and/or affecting the at least one smooth muscle leads to an effect, for example an indirect effect on the blood flow, for example as described in FIG. 3.

According to some exemplary embodiments, the delivered electric field affects arousal of at least one genital organ, at block 617. In some embodiments, affecting arousal of a genital organ refers to increasing shape and/or size of the genital organ, optionally by increasing blood flow and/or volume and/or reducing blood leakage from the genital organ.

According to some exemplary embodiments, the genital arousal following the delivery of the electric field is assessed, at block 619. In some embodiments, at least one physiological parameter associated with sexual arousal and/or activation levels of the sympathetic and/or parasympathetic systems, is assessed at block 619.

According to some exemplary embodiments, the parameter values are optionally modified at block 621, for example according to the assessment results. In some embodiments, the parameter values, for example the electric field parameter values are modified in order achieve a predetermined arousal effect in the subject.

According to some exemplary embodiments, electric filed is optionally delivered to the subject with the modified parameter values.

Reference is now made to FIG. 6C, depicting a detailed feedback process, according to some exemplary embodiments of the invention.

According to some exemplary embodiments, at least one physiological parameter is measured at block 640. In some embodiments, baseline values of the at least one physiological parameter are measured. In some embodiments, the at least one physiological parameter is measured prior to the delivery of an electric field, for example to determine a state of the autonomic nervous system. In some embodiments, the at least one physiological parameter is measured in a female subject. In some embodiments, the at least one physiological parameter comprises at least one of HRV, VPA, CBV, LPA, and/or SCL.

According to some exemplary embodiments, the at least one physiological parameter is measured using at least one sensor of the sexual arousal device or at least one sensor in communication with the sexual arousal device. Alternatively, the at least one physiological parameter is measured by at least one sensor of a device which is not in communication with the sexual arousal device.

According to some exemplary embodiments, baseline HRV is measured using LPA or third-party devices or mobile apps. In some embodiments, the baseline measurements are performed during resting state. Alternatively, current level of HRV is compared with average measurements of relevant population (sex, age, physical state, etc.). Alternatively or additionally, baseline levels of VPA or CBV or LPA or a combination thereof are measured. Alternatively or additionally, baseline levels of SCL are measured.

According to some exemplary embodiments, a relation between the measured values of the at least one parameter and predetermined stored values is determined at block 642. In some embodiments, the predetermined stored clause indicates a desired or a target state of the subject, for example a target state of the autonomic nervous system of the subject, for example a female subject.

According to some exemplary embodiments and without being bound by any theory, a female subject diagnosed with an arousal disorder has elevated sympathetic activity, therefore the measurements would reveal overly reduced HR variability. In some embodiments, the required stimuli would then aim to reduce sympathetic activity and elevate parasympathetic activity. Alternatively, some female subjects have highly reduced sympathetic activity, and therefore, their measurements would reveal high HRV values. In some embodiments, if HRV levels are high that a predetermined value, the required stimuli would then aim to elevate sympathetic activity and reduce parasympathetic activity.

According to some exemplary embodiments, if the measured HRV is higher than a predetermined value at block 644 or a target value, for example a normal value, an electric field is delivered at block 646 to increase sympathetic activity and/or reduce parasympathetic activity. In some embodiments, the electric field is delivered at block 646 with parameter values, for example frequency, pulse width and/or intensity sufficient to increase activity of at least one sympathetic nerve, and/or to decrease activity of at least one parasympathetic nerve. In some embodiments, the electric field is delivered at blocks 646 and 648 with a frequency in a range of 0.5 Hz-50 Hz, for example 0.5 Hz-10 Hz, 0.5 Hz-5 Hz, 2 Hz-8 Hz, 0.5 Hz-5 Hz, or any intermediate, smaller or larger range of frequency values. In some embodiments, the electric field is delivered at blocks 646 and 648 with a pulse width in a range of 10 μs-600 μs, for example 50 μs-400 μs, 20 μs-100 μs, 50 μs-100 μs, 50 μs-200 μs, or any intermediate, smaller or larger range of pulse widths. In some embodiments, the electric field is delivered at blocks 646 and 648 with an intensity in a range of 0 mA-100 mA, for example 0 mA-40 mA, 0 mA-20 mA, 5 mA-30 mA, 10 mA-40 mA, or any intermediate, smaller or larger range of values.

According to some exemplary embodiments, if the measured HRV is lower than a predetermined value at block 650 or a target value, for example a normal value, an electric field is delivered at block 652 to reduce sympathetic activity and/or increase parasympathetic activity. In some embodiments, the electric field is delivered at block 652 with parameter values, for example frequency, pulse width and/or intensity sufficient to decrease activity of at least one sympathetic nerve, and/or to increase activity of at least one parasympathetic nerve. In some embodiments, the electric field is delivered at blocks 652 and 654 with a frequency in a range of 50 Hz-200 Hz, for example 50 Hz-100 Hz, 50 Hz-150 Hz, 70 Hz-120 Hz, 80 Hz-150 Hz, or any intermediate, smaller or larger range of frequency values. In some embodiments, the electric field is delivered at blocks 652 and 654 with a pulse width in a range of 10 us-600 μs, for example 50 μs-400 μs, 20 μs-100 μs, 50 μs-100 μs, 50 μs-200 μs, or any intermediate, smaller or larger range of pulse widths. In some embodiments, the electric field is delivered at blocks 652 and 654 with an intensity in a range of 0 mA-100 mA, for example 0 mA-40 mA, 0 mA-20 mA, 5 mA-30 mA, 10 mA-40 mA, or any intermediate, smaller or larger range of values.

According to some exemplary embodiments, following delivery of electric field at block 652 or block 646, the at least one physiological parameter, for example VPA, CBV and/or LPA is measured. In some embodiments, a relation between the physiological parameter values measured following electric field delivery, and the physiological parameter values measured at block 640 is determined. In some embodiments, the relation is determined for example to detect changes in the activity of the autonomic nervous system following the delivery of the electric field.

According to some exemplary embodiments, VPA, CBV, and/or LPA is measured at block 656. In some embodiments, the values measured at block 656 are compared to values measured prior to the electric field delivery, for example at block 640. In some embodiments, if VPA, CBV, and/or LPA levels have been elevated at block 658, then the delivery of the electric field is continued at block 660 with the previously parameter values used at blocks 652 and 654.

According to some exemplary embodiments, if VPA, CBV, and/or LPA levels have been remained unchanged or within a range of up to 5%, up to 3%, up to 1%, up to 0.5% or any intermediate, smaller or larger value at block 662 relative to the previously measured values, then the delivery of the electric field is continued at block 664 with intensity levels that are higher than the previously parameter values used at blocks 652 and 654.

According to some exemplary embodiments, if VPA, CBV, and/or LPA levels have been reduced at block 666 relative to the previously measured values, then the delivery of the electric field is continued at block 668 with different parameter values relative to the parameter values previously used at blocks 652 and 654.

According to some exemplary embodiments, in male subjects, the electric field is delivered with parameter values, for example frequency, pulse width and/or intensity sufficient to elevate parasympathetic activity. In some embodiments, in male subjects, the electric field is delivered with a frequency in a range of 50 Hz-200 Hz, for example 50 Hz-100 Hz, 50 Hz-150 Hz, 70 Hz-120 Hz, 80 Hz-150 Hz, or any intermediate, smaller or larger range of frequency values. In some embodiments, in male subjects the electric field is delivered with a pulse width in a range of 10 μs-600 μs, for example 50 μs-400 μs, 20 μs-100 μs, 50 μs-100 μs, 50 μs-200 μs, or any intermediate, smaller or larger range of pulse widths. In some embodiments, in male subject the electric field is delivered with an intensity in a range of 0 mA-100 mA, for example 0 mA-40 mA, 0 mA-20 mA, 5 mA-30 mA, 10 mA-40 mA, or any intermediate, smaller or larger range of values.

According to some exemplary embodiments, male subjects diagnosed with anxiety or performance dysfunction would exhibit high sympathetic activity, and/or low HRV levels, and optionally certain degree of erectile dysfunction. In some embodiments, electric field delivery will have frequency in a range of 50 Hz-200 Hz, pulse width in a range of 10 μs-600 μs, and/or with intensity in a range of 0 mA-100 mA, for a time period starting up to 30 minutes before sexual intercourse and until end of sexual intercourse, or till effect onset.

### Exemplary Activation/Deactivation

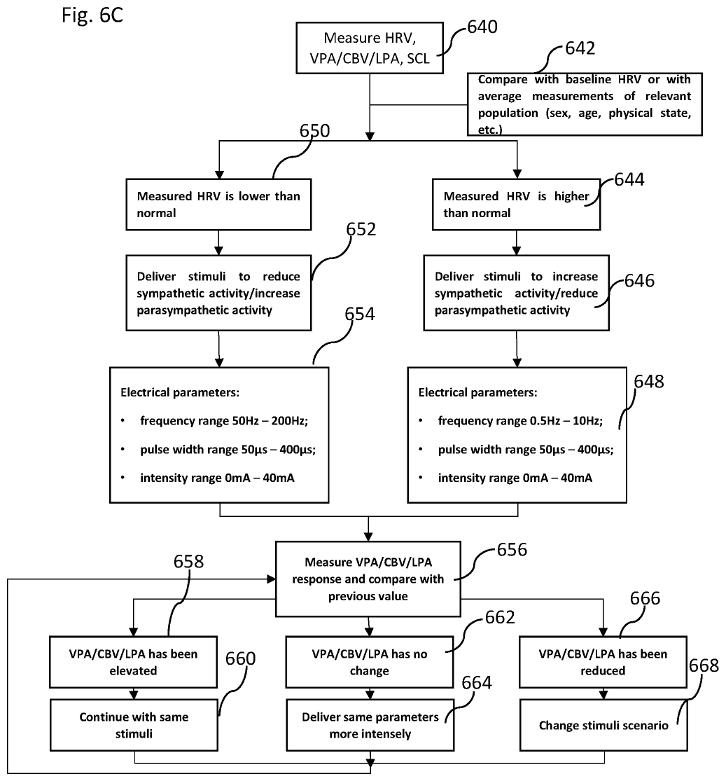

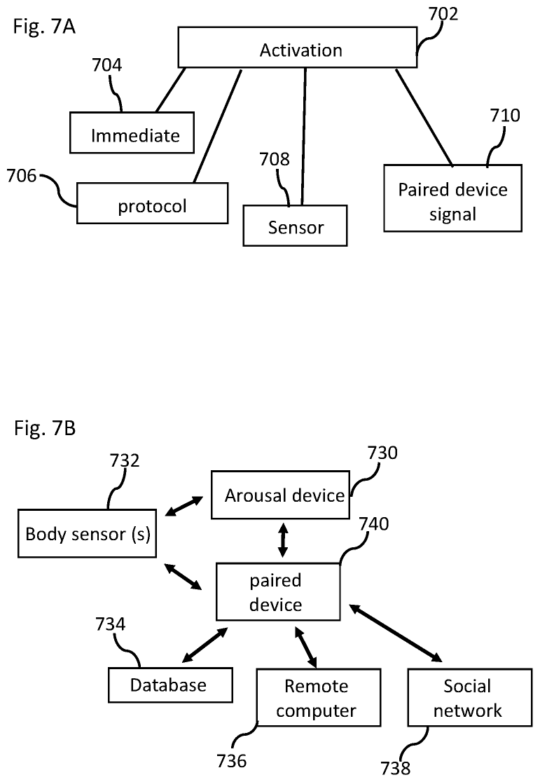

Reference is now made to FIG. 7A, depicting activation and/or deactivation options of the sexual arousal device, according to some exemplary embodiments of the invention.

According to some exemplary embodiments, the sexual arousal device is activated prior to and/or during sexual intercourse. Additionally, the sexual arousal device is deactivated when the sexual intercourse stops or completed.

According to some exemplary embodiments, the sexual arousal device is activated, for example starts to deliver an electric field or initiates an automatic process for delivery of the electric field at block 702. In some embodiments, the device is activated immediately at block 704. In some embodiments, the device is activated immediately for example when the power supply, for example at least one battery is electrically connected to the control, circuitry and/or to a pulse generator of the device. In some embodiments, the device is activated immediately when an isolator preventing an electrical connection between the power supply and the control circuitry and/or the pulse generator is removed.

According to some exemplary embodiments, the sexual arousal device is activated according to predetermined protocol at block 706. In some embodiments, the device is activated according to at least one activation protocol stored in a memory of the device. In some embodiments, the at least one activation protocol or parameters thereof are selected using at least one user interface of the device. Alternatively, the at least one activation protocol or parameters thereof are selected automatically by the device, for example based on measurements performed by the device, for example based on measurements of at least one physiological parameter.

According to some exemplary embodiments, the sexual arousal device is activated based on a signal received from at least one sensor, at block 708. In some embodiments, the sexual arousal device is activated based on a signal received from at least one sensor of the device, and/or at least one sensor in communication with the device. In some embodiments, the sensor comprises a heat sensor, a proximity sensor, a sensor configured to measure heart rate, blood flow, blood pressure and/o skin conductance.

According to some exemplary embodiments, the sexual arousal device is activated by a signal received from a paired device, at block 710. In some embodiments, the paired device comprises a remote control, a virtual assistance device, a cellular device optionally including a stored activation application of the arousal device, a remote computer and/or a paired sensor.

### Exemplary Sexual Arousal Device Communication

According to some exemplary embodiments, the sexual arousal device is configured to communicate, for example to transmit and/or receive data, with at least one device. Reference is now made to FIG. 7B, depicting communication of the arousal device with at least one additional device, according to some exemplary embodiments of the invention.

According to some exemplary embodiments, a sexual arousal device 730 is in communication with at least one additional device, optionally via wireless communication. In some embodiments, the device 730 is in communication with at least one body sensor 732. In some embodiments, the body sensor 732 comprises a sensor configured to sense at least one physiological parameter, for example heart rate, blood flow, blood volume, skin conductance, heat, and/or proximity. In some embodiments, the at least one body sensor is a sensor positioned on a body of a subject wearing the sexual arousal device. Alternatively, the at least one body sensor is a sensor attached to a body of partner of the sexual intercourse. In some embodiments, the arousal device 730 is activated, deactivated and/or changes activation state and/or activation parameter values, according to a signal received from at least one body sensor 732.

According to some exemplary embodiments, the arousal device 730 is in communication with at least one paired device 740. In some embodiments, the paired device 740 comprises a cellular device, a mobile device, a smartphone, a smartwatch, and/or a virtual assistant device. In some embodiments, the arousal device transmits and/or received signals from the paired device. In some embodiments, the arousal device 730 is activated, deactivated and/or changes activation state and/or activation parameter values, according to a signal received from the paired device 740. In some embodiments, the arousal device 730 transmits log files and/or measurements of at least one physiological parameter to the paired device 740.

According to some exemplary embodiments, the paired device 740 is in communication with at least one sensor, for example with a body sensor 732. In some embodiments, the paired device 740 transmits measurements of the at least one sensor to the arousal device 730. Alternatively or additionally, the paired device processes the signals received from the at least one sensor, and optionally transmits the processing results to the arousal device 730.

According to some exemplary embodiments, the paired device 740 is in communication with at least one database 734, for example a database on a cloud storage. In some embodiments, the paired device 740 transmits signals received from the arousal device 730 and/or from the at least one sensor, for example body sensor 732 to the database 734. Optionally, at least one algorithm, look up table or a software installed in the database 734 processes the received signals and generates one or more output signals to be delivered to the paired device 740 and/or to the arousal device 730. In some embodiments, the database receives one or more signals indicating a state of a subject before and/or following the electric field delivery, and transmits an out signal including recommendations and/or changes in electric field delivery parameters or protocol, to the paired device 740 and/or to the arousal device 730.

According to some exemplary embodiments, the paired device 740 is in communication with at least one remote computer 736, for example as described with respect to the database 734. According to some exemplary embodiments, the paired device 740 is in communication with at least one social network. In some embodiments, the paired device provides updates to at least one social network profile or account based on signals received from the arousal device 730, for example based on signals received during sexual intercourse or self-pleasuring. In some embodiments, the signals indicate at least one of body posture, orientation, movement, sexual intercourse duration, self-pleasuring duration, arousal level of a subject wearing the arousal device 730 and/or arousal level of a partner of the subject.

### Exemplary Interaction Between at Least Two Subjects

According to some exemplary embodiments, two or more arousal devices are in communication with each other. In some embodiments, the two or more arousal device synchronize, activate and/or deactivate at least one device of the two or more arousal devices according to signals received from a different device.

According to some exemplary embodiments, prior to sexual intercourse, a first sexual arousal device is attached to a skin surface of a first subject, and a second sexual arousal device is attached to a skin surface of a second subject. In some embodiments, when the first and second subjects get closer to each other, at least one or both of the sexual arousal devices are activated, for example start to delivery an electric field. Alternatively or additionally, when the first and second subjects and/or the two sexual arousal devices are close to each other, for example at a distance shorter than 2 meters, 2.5 meters, 1 meter, 0.5 meter or any intermediate, smaller or larger distance, the devices change at least one of activation state, parameters of the electric field delivery, a treatment protocol, optionally according to the distance between the two devices and/or according to signals received from at least one device of the two sexual arousal devices.

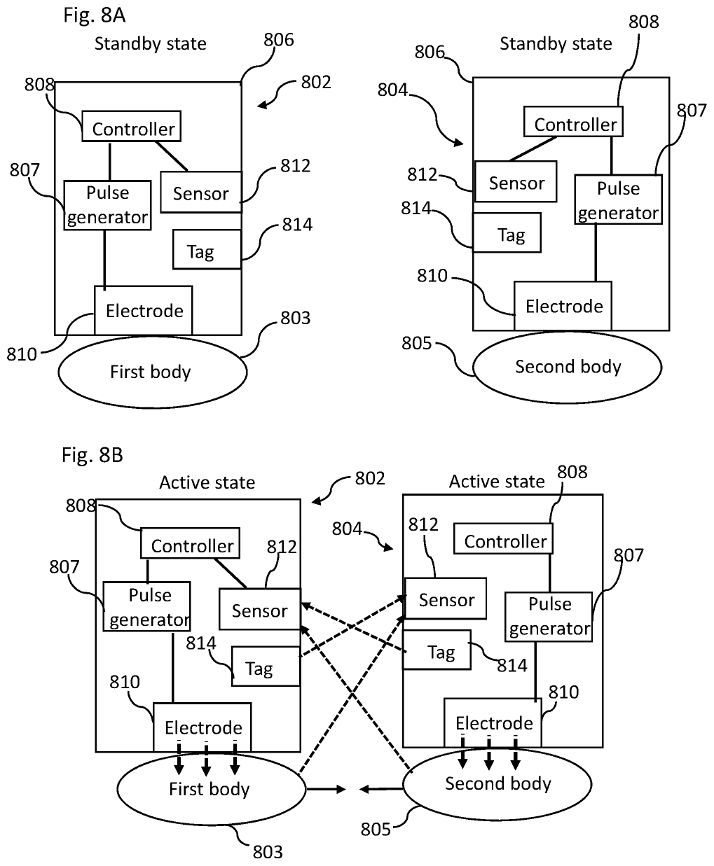

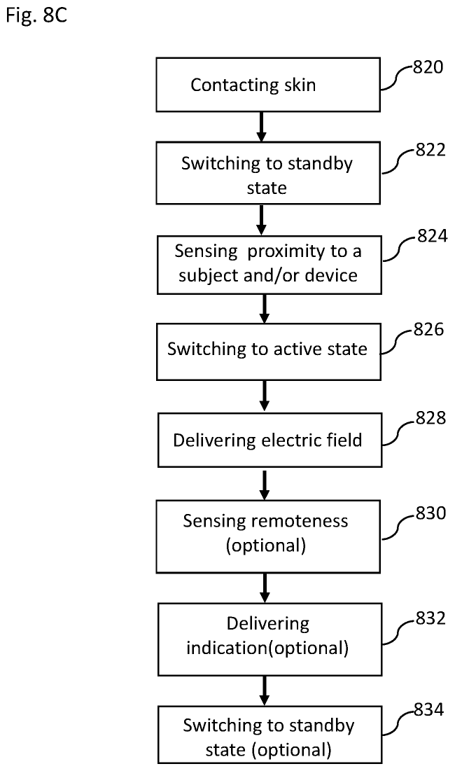

Reference is now made to FIGS. 8A and 8B, depicting two arousal devices, each on a different subject, according to some exemplary embodiments of the invention.

According to some exemplary embodiments, a first arousal device 802 is attached to a first subject, for example a male subject or a female subject. In some embodiments, a second arousal device 804 is attached to a second subject, for example a male subject or a female subject.

According to some exemplary embodiments, each of the arousal devices 802 and 804 comprise a housing 806 shaped and sized to be attached to a body, for example a skin surface of the subject, for example as described with regard to body 404 shown in FIG. 4A. In some embodiments, each of the devices comprises a control circuitry, for example controller 808 in communication with at least one pulse generator 807 and electrode 810, for example two or more electrodes. In some embodiments, the controller 808 is configured to signal the pulse generator 807 to generate and deliver an electric field to the electrode 810, and transcutanously to a body contacting the electrode 810.

According to some exemplary embodiments, at least one or each of the arousal devices 802 and 804 comprises at least one sensor, for example sensor 812 configured to sense proximity, for example proximity to a different device, or to a tag of a device, for example tag 814. In some embodiments, the sensor 812 comprises a RFID reader, and the tag 814 comprises a RFID tag.

Reference is now made to FIG. 8C, depicting a process for changing an activation state of an arousal device based on interaction with a different arousal device, according to some exemplary embodiments of the invention.

According to some exemplary embodiments, arousal devices, for example device 802 and 804 are placed in contact with a skin of different subjects. In some embodiments, each device is placed in contact with a skin surface of a different subject, for example a male subject and a female subject, or a male subject and a male subject, or a female subject and a female subject. In some embodiments, each device is placed in contact with a skin surface located at close proximity to a genital organ, for example at a close proximity to the clitoris or at a close proximity to a penis, for example to a penis base.

According to some exemplary embodiments, at least one device or each of the devices, for example devices 802 and 804 are switched to a standby state. In some embodiments, the devices are switched to a standby state, for example as shown in FIG. 8A, or are at a standby state, prior to sexual intercourse. In some embodiments, in a standby state, the device controller, for example controller 808 is configured to receive signals from the sensor 812, for example without delivery of an electric field through electrode 810 to a body.

According to some exemplary embodiments, a proximity to a subject and/or to an additional device is detected at block 824. In some embodiments, when the two subjects get closer to each other, for example as shown in FIG. 8B, the two devices, for example devices 802 and 804 are in relative proximity to each other. In some embodiments, at least one device or both of the devices sense a proximity to a subject or to at least one additional device. In some embodiments, the device senses proximity to an additional device when a distance between the two devices is smaller than 2 meters, for example smaller than 1.5 meters, smaller than 1 meter, smaller than 0.5 meter or any intermediate, smaller or larger distance between the two devices.

According to some exemplary embodiments, at least one device of devices 802 and 804 senses proximity to a different device, for example as shown in FIG. 8B, based on signals received from the at least one sensor 812. Alternatively, at least one device of devices 802 and 804 senses proximity to a different device, for example as shown in FIG. 8B, based on identification of a tag, for example tag 814 associated with the different device.

According to some exemplary embodiments, proximity of a device to a subject is detected, for example, based on a tag associated with a subject, and a sensor of the device configured to read the tag. Alternatively, a presence of a subject is detected based on contact between the subject and the device, and/or heat of the subject. In some embodiments, the subject heat is detected by the at least one sensor of the device or at least one sensor, for example an optic sensor or a thermal camera in communication with the device.

According to some exemplary embodiments, for example as shown in FIG. 8B, when proximity to a subject or a different device is detected, at least one of the devices, for example devices 802 or 804 is switched to an active state, at block 826. In some embodiments, switching to an active state comprises delivering an electric field to a tissue of the subject by at least one electrode. In some embodiments, both devices start to deliver an electric field when they are at a close distance to each other, for example at a distance smaller than 2 meters. In some embodiments, when the two devices are at a close distance relative to each other, the controller 808 signals the pulse generator 807 to generate an electric field and to deliver the electric field to the at least one electrode 810, for example as shown in FIG. 8B.

According to some exemplary embodiments, at least one device optionally senses remoteness of the additional device or the subject, at block 830. In some embodiments, sensing remoteness comprises sensing that a distance between the device and a different device is larger than 2 meters, for example larger than 2.5 meters, larger than 3 meters or any intermediate, smaller or larger distance. In some embodiments, the device senses remoteness based on signals received from the sensor 812. Alternatively or optionally, the device senses remoteness when signals received from the sensor 812 are stopped.

According to some exemplary embodiments, an indication is optionally delivered at block 832. In some embodiments, the indication, for example a human detectable indication is delivered to a subject wearing the device. In some embodiments, the indication is optionally delivered when sensing remoteness, for example before stopping the electric field delivery. In some embodiments, the indication is delivered by vibration. In some embodiments, the indication is optionally delivered to a device paired and/or in communication with the sexual arousal device, for example to a mobile device, a cellular device, a wearable device, for example a smartwatch. Optionally, the paired device delivers a human detectable indication to a subject based on signals and/or indications received from the sexual arousal device.

According to some exemplary embodiments, at least one of the sexual arousal devices is optionally switched to a standby state at block 834. In some embodiments, an activation state of the device is optionally switched to a standby state when remoteness is sensed at block 830. In some embodiments, when the device activation state is switched to a standby state, electric field delivery is stopped.

### Exemplary Device Structure

According to some exemplary embodiments, a sexual arousal device is formed as a skin patch, having at least one surface shaped to be attached to a skin surface of a subject. In some embodiments, the device comprises at least two electrodes positioned at the at least one surface to be positioned in contact with the skin surface. In some embodiments, an electrical circuitry comprising a control circuitry, for example a controller, a power supply, for example at least one battery, and optionally a pulse generator, is integrated with the skin patch. Optionally, at least a portion of the electrical circuitry is exposed through at least one surface of the device, for example a surface of the device which is opposite to the surface contacting the skin surface.

According to some exemplary embodiments, the skin patch comprises an inwardly curved opening, for example a v shaped opening, which has an opening width suitable to position at least one electrode of the device on each side of a genital organ, for example the clitoris, the labia majora, the penis, the base of a penis. Optionally, the inwardly curved opening is shaped and sized to receive the genital organ within the inwardly curved opening while electrodes of the device surround, at least partly the genital organ. Alternatively the device is long enough to position at least one electrode on each side of the genital organ.

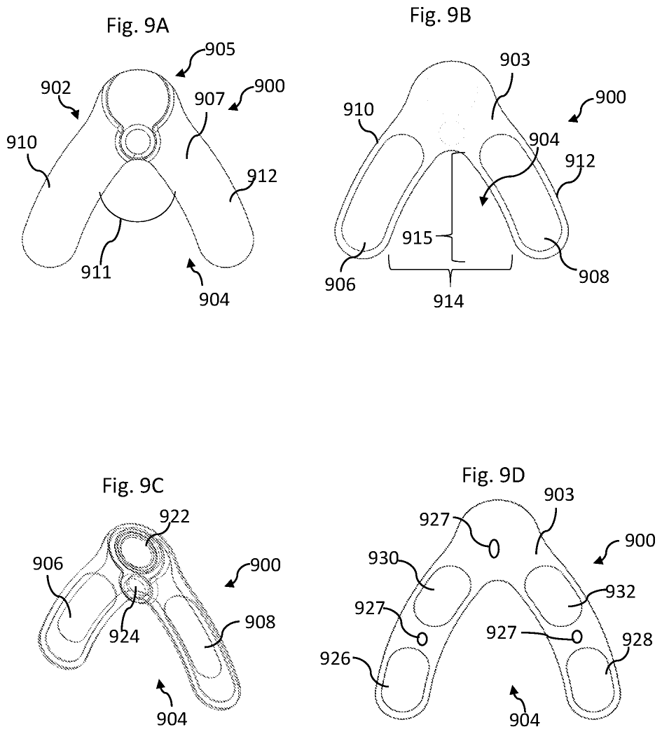

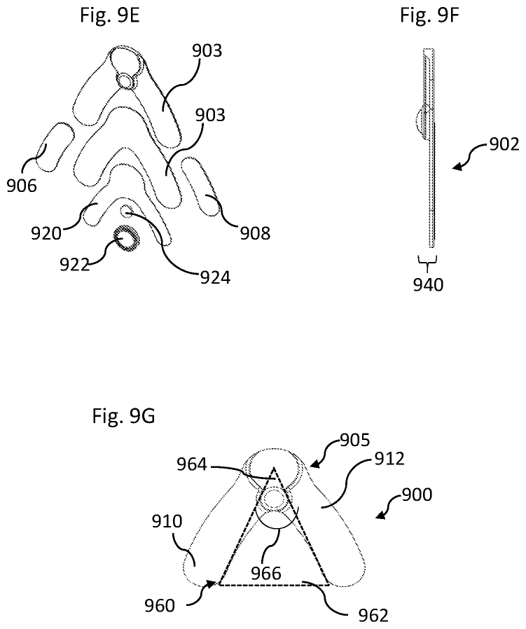

Reference is now made to FIGS. 9A-9G, depicting a sexual arousal device having an inwardly curved opening, according to some exemplary embodiments of the invention.

According to some exemplary embodiments, a sexual arousal device 900 comprises a body 902, having an inwardly curved opening 904. In some embodiments, a first surface of the body, for example a skin contacting surface 903 shown in FIG. 9B is shaped and sized to be attached to a skin surface of a subject. In some embodiments, the surface 903 is smooth, and optionally planar, for example not to damage the skin surface. Alternatively, the surface 903 comprises two or more bulges shaped and sized to allow contact between the surface 903 and the skin surface through a hair layer.

According to some exemplary embodiments, the device comprises a second surface 907 opposite to the skin contacting surface 903. In some embodiments, the second surface 907 is configured to include a user interface, for example one or more activation buttons and/or visual indicators. In some embodiments, the surface 907 is a non-planar surface comprising one or more bulges or protrusions, optionally preformed, to accommodate at least part of a user interface positioned within the body 902.

According to some exemplary embodiments, the device 900 comprises 2 or more electrodes, for example electrodes 906 and 908, configured to contact the skin surface. In some embodiments, the electrodes 906 and 908 are located in the surface 903. Alternatively, the electrodes 906 and 908 are located on the surface 903.

In some embodiments, a surface area of each of the electrodes 906 and 906 is in a range of 20 mm²-500 mm², for example 20 mm²-50 mm², 30 mm²-100 mm², 50 mm²-200 mm², 100 mm²-300 mm², or any intermediate, smaller or larger range of values.

According to some exemplary embodiments, the body 902 comprises a central region 905 and at least two extensions, for example extensions 910 and 912 connected to the central region 905. In some embodiments, the at least two extensions extend from the central regions 905. In some embodiments, the extensions are fixed, for example stationary with respect to the central region 905. Alternatively, at least one of the extensions 910 and 912 is movable relative to the central region 905 and/or relative to at least one different extension. In some embodiments, an angle 911 between two extensions of the at least two extensions is adjustable, for example to define a size and/or a width of an opening between the two extensions. Alternatively, the angle 911 is fixed. In some embodiments, the angle 911 has a value in a range between 70 degrees and 15 degrees, for example a value in a range between 50 degrees and 20 degrees, a value between 45 degrees and 25 degrees, a value between 40 degrees and 20 degrees, or any intermediate, smaller or larger range of values. In some embodiments, the angle 911 between the two extensions, for example wings, 910 and 912, is selected in order to position the extensions on both sides of the vagina, for example to allow specific electrification of tissue located closer to the vagina. In some embodiments, the device is configured to electrify tissue on one or both sides of the vagina. In some embodiments, the electric field generated by the device is delivered with parameter values selected to electrify tissue located at a distance of up to 5 cm, for example up to 4 cm, up to 3 cm, up to 2 cm or any intermediate smaller or larger lateral distance from a side of the vagina.

According to some exemplary embodiments, each electrode of the at least two electrodes 906 and 908 is located on a different extension. Optionally, the extensions 910 and 912 define the inwardly curved opening 904.

According to some exemplary embodiments, a width 914 of the opening is in a range of 2 cm to 20 cm, for example 2 cm-5 cm, 3 cm-8 cm, 4 cm-10 cm, 5 cm-15 cm, 10 cm-20 cm or any intermediate, smaller or larger distance. In some embodiments, a distance between two electrodes located at opposite ends of the device 900, for example electrodes 906 and 908, is in a range of 3-15 cm, for example 5 cm-10 cm, 7 cm-12 cm, 9 cm-15 cm or any intermediate, smaller or larger distance. In some embodiments, a minimal length 915 of the opening 904 is in a range of 1 cm-10 cm, for example 1 cm-5 cm, 3 cm-8 cm, 5 cm-10 cm or any intermediate, smaller or larger value.

According to some exemplary embodiments, the device 900 comprises an electronic circuitry electrically connected to the at least two electrodes 906 and 908. In some embodiments. the electronic circuitry comprises a printed circuit board 920, for example a flexible circuit board within body 902, operationally coupled to the at least two electrodes. Additionally or alternatively, the device 902 comprises a control circuitry, a user interface and power supply, for example at least one battery 922. In some embodiments, the user interface, comprises at least one activation button 924 configured to receive an input signal.

According to some exemplary embodiments, the electronic circuitry delivers an electric field to the at least two electrodes, for example when receiving an input signal from the user interface, for example the activation button 924. In some embodiments, the device comprises a pulse generator in said body. In some embodiments, the control circuitry is configured to signal the pulse generator to generate and deliver an electric field from said at least two electrodes 906 and 908 in response to an input signal received from the user interface, for example the activation button 924.

According to some exemplary embodiments, the electronic circuitry is integrated within the body 902. Alternatively, the electronic circuitry is attached, for example within a housing to the surface 907.

According to some exemplary embodiments, the at least two electrodes comprise 4, 6, 8 or any number or electrode pairs. In some embodiments, the at least two electrodes are arranged in pairs, where each electrode of every pair is located on a different extension of the device 900, and optionally at a different side of the opening 904, for example electrodes 926 and 928, and electrodes 930 and 932 shown in FIG. 9D.

According to some exemplary embodiments, at least part of the body 902 or the entire body 902 is flexible, for example to conform to anatomy of the mons pubis of a female subject. Alternatively or additionally, at least part of the body 902 or the entire body 902 is flexible and/or bendable, for example to conform to the movement of a subject and/or to prevent discomfort during sexual intercourse. In some embodiments, at least a portion of the body 902 has a minimal radius of tortuosity of about 0.5 mm, for example a minimal radius of tortuosity of about 1 mm, a minimal radius of tortuosity of about 2.5 mm, a minimal radius of tortuosity of about 3 mm, a minimal radius of tortuosity of about 3.5 mm, a minimal radius of tortuosity of about 4 mm, or any intermediate, smaller or larger value. In some embodiments, that at least one flexible portion of the body has a minimal bending radius larger than 0.5 mm, 1 mm, 2 mm, 3 mm, or any intermediate, smaller or larger value.

According to some exemplary embodiments, the body 902, for example opening 904 is configured not to block a vagina of a female subject and/or to prevent penetration through the vagina.

According to some exemplary embodiments, for example as shown in FIG. 9F, the body 902 is thin, and has a maximal thickness 940 of up to 15 mm, for example up to 12 mm, up to 10 mm, up to 6 mm, up to 1 mm or any intermediate, smaller or larger value.

According to some exemplary embodiments, energy in the power supply, for example at least one battery 922 is sufficient for delivery of an electric field for up to 2 hours, for example up to 1.5 hours, up to 1 hour, up to 45 minutes, up to 30 minutes, up to 15 minutes, up to 10 minutes or any intermediate, smaller or larger time period. In some embodiments, the power supply is rechargeable. Alternatively or additionally, the power supply is removable and optionally replaceable.

According to some exemplary embodiments, the device 900 is disposable. Alternatively, at least part of the electronic circuitry is reusable, for example at least one of the power supply, the control circuitry, the user interface, the PCB, the at least two electrodes. Optionally, the device 900 is reusable, for example when the power supply is rechargeable and/or replaceable. In some embodiments, the at least two electrodes 906 and 908 are disposable. Optionally, the at least two electrodes 906 and 906 are removably coupled to the body 902, for example to the skin contacting surface 903. Optionally, the device 900 is connectable to a charger or a docking station, for example to allow charging and/or communication with a computer, cellular phone or a mobile device. In some embodiments, the device 900 comprises at least one charging and/or communication port, for example to allow the charging and/or communication.

According to some exemplary embodiments, the body 902 is sealed, for example to prevent penetration of liquids into the body 902.

According to some exemplary embodiments, for example as shown in FIG. 9D, the device 900 comprises at least one sensor 927, for measuring at least one physiological parameter of the subject body. In some embodiments, the at least one physiological parameter comprises skin conductance, heat, blood flow, blood volume and/or heart rate. In some embodiments, at least part of the sensor 927 is located at the skin contacting surface 903, for example to allow contact or proximity between the at least one sensor and the skin surface of the subject. Alternatively or additionally, at least part of the sensor 927 is located on the skin contacting surface 903. In some embodiments, the at least one sensor comprises a photoplethysmography sensor, configured to measure a change in blood volume of a blood vessel. In some embodiments, the at least one sensor is operationally coupled to the control circuitry, for example to allow treatment feedback, for Example as described in FIGS. 6B and 6C.

According to some exemplary embodiments, for example as shown in FIG. 9G. an opening 960 between the two extensions 910 and 912 is shaped and sized to allow insertion of a virtual triangle 962, such that when the triangle apex 964 overlaps with the central region 905 and the triangle base corners contact the extensions 910 and 912, for example an inner surface of the extensions 910 and 912, at a distance of up to 2 cm from the opening 960 entrance, a head angle 966 of the triangle 962 is in a range between 15 degrees and 70 degrees, for example in a range between 15 degrees and 50 degrees, in a range between 20 degrees and 60 degrees, or any intermediate, smaller or larger range of values.

### Exemplary Device Positioning

According to some exemplary embodiments, the device, for example the body of the device is configured to be attached at least partly to a mons pubis of a female subject. Alternatively, the device, for example the body of the device is configured to be attached at least partly to a skin surface superior to a base of a penis.

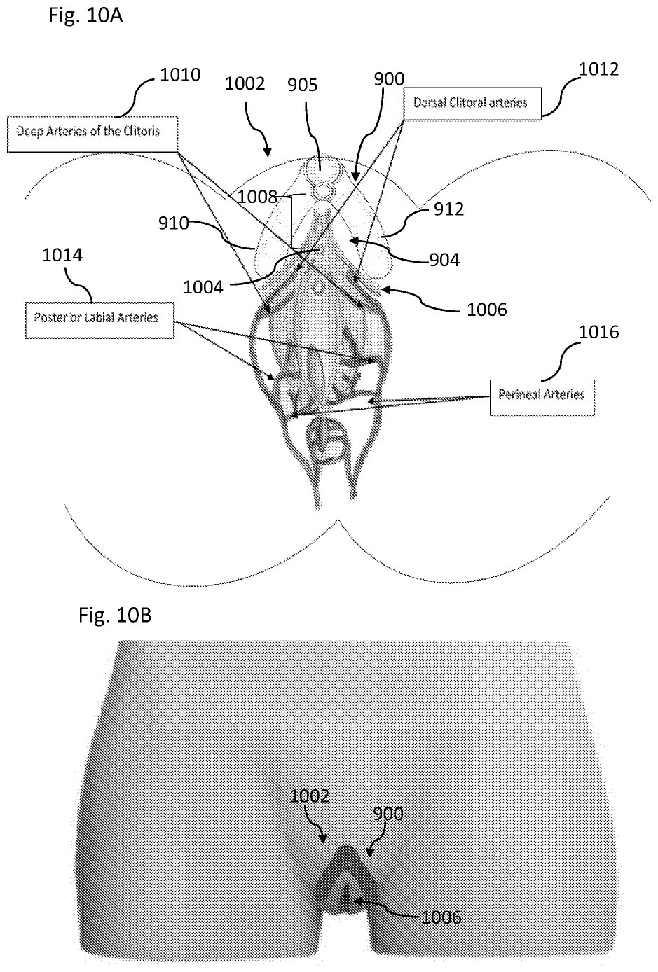

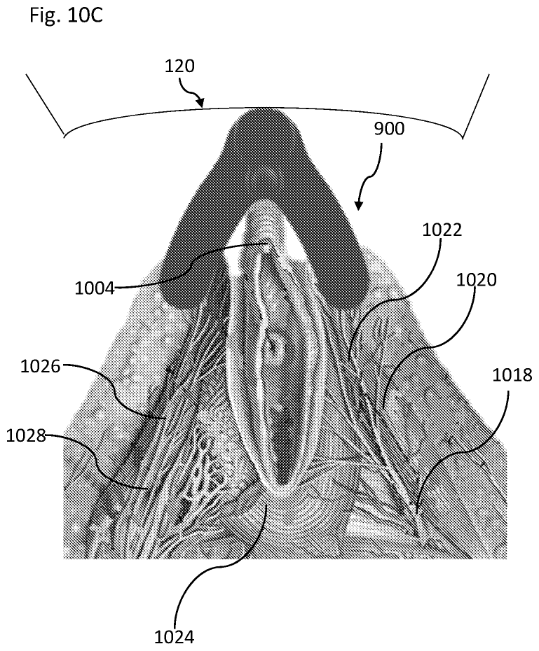

Reference is now made to FIGS. 10A-10C, depicting a position of a sexual arousal device, according to some exemplary embodiments of the invention.

According to some exemplary embodiments, a device 900 is configured to be attached, for example a skin contacting surface of the device, at least partly to a mons pubis 1002 of a female subject. In some embodiments, a central portion 905 of the device 900 is attached to the mons. In some embodiments, the device 900 is attached, at least partly, to a skin surface of the mons pubis 1002.

According to some exemplary embodiments, attaching the central portion 905 of the device 900 to a skin surface of the mons pubis 1002, positions the clitoris 1004 within the opening 904. and/or each extension of the device, for example extensions 910 and 910, at opposite sides of the clitoris 1004 and/or the labia majora 1006. Alternatively or additionally, attaching the device 900 at least partly to a skin surface of the mons pubis 1002, positions the clitoris 1004 within the opening 904, and at least two electrode of the device 900, each on a different side, optionally opposite side, of the clitoris 1004.

According to some exemplary embodiments, the device 900 is attached to a skin surface near a genital organ, for example the clitoris 1004, at a minimal distance 1008 of up to 10 cm, for example up to 7 cm, up to 5 cm, up to 3 cm or any intermediate, smaller or larger distance, from the clitoris 1004. In some embodiments, attaching the device 900 to the skin surface near the genital organ, positions the genital organ between at least two electrodes of the device 900, for example to direct an electric field delivered between the at least two electrodes and transcutanously to the skin tissue towards at least one tissue optionally associated with the genital organ. In some embodiments, the tissue comprises a nerve, for example a nerve of the autonomic system, a muscle and/or a blood vessel, optionally associated with the genital organ.

According to some exemplary embodiments, for example as shown in FIG. 10A, the tissue affected by the delivered electric comprises arteries delivering blood to the clitoris, for example deep arteries of the clitoris 1010 and/or dorsal clitoral arteries 1012. Alternatively or additionally, the delivered electric field affects one or more arteries associated with the Labia, for example arteries delivering blood to the Labia, for example posterior labial arteries 1014. Alternatively or additionally, the delivered electric field affects one or more arteries of the perineum, for example one or more perineal arteries 1016.

According to some exemplary embodiments, for example as shown in FIG. 10C, the delivered electric field affects one or more nerves, for example the posterior labial nerve 1018 and/or the dorsal nerve of the clitoris 1026. Alternatively or additionally, the delivered electric field affects the ischicavernosus muscle 1020 and/or the bulbospongious muscle 1024. Additionally or alternatively, the delivered electric field affects the internal pudental artery 1028.

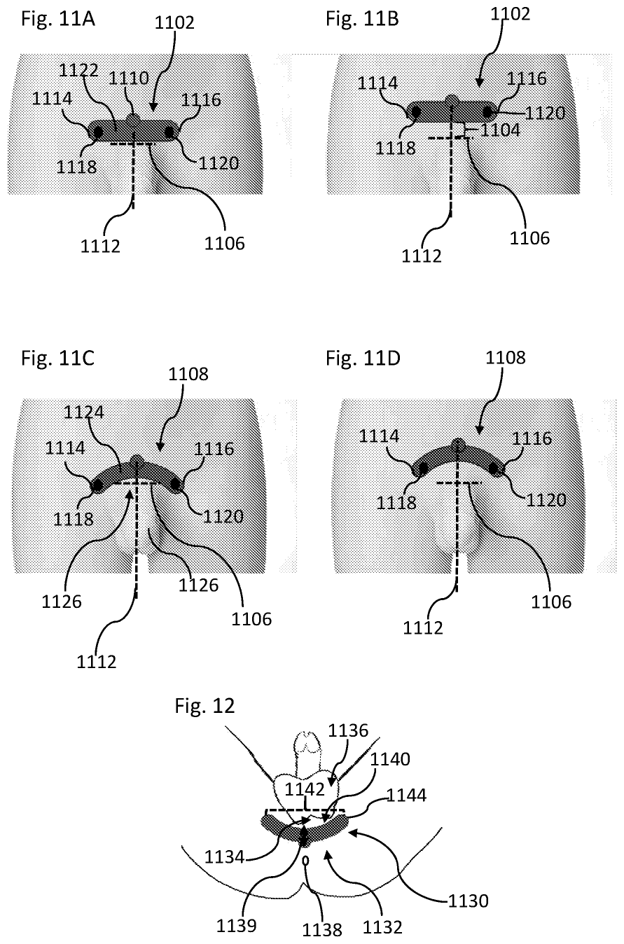

Reference is now made to FIGS. 11A-11D, depicting positioning a sexual arousal device on a skin surface of a male subject, according to some exemplary embodiments of the invention.

According to some exemplary embodiments, a sexual arousal device, for example devices 1102 and 1108 are attached to a skin surface of a male subject body region which is superior to the penis, for example superior to a base of the penis.
In some embodiments, the device is attached to a skin surface of a body region located at a minimal distance 1104 which is shorter than 10 cm, for example shorter than 8 cm, 5 cm, 3 cm, 1 cm or any intermediate, smaller or larger distance from a base 1106 of a penis. In some embodiments, the sexual arousal device is attached to a skin surface of body region which is ventral to pubic symphysis.

According to some exemplary embodiments, a central portion of the device, for example central portion 1110 is aligned with a midline 1112 of the penis. In some embodiments, aligning the central portion 1110 with the midline positions two lateral ends 1114 and 1116 on both sides of a penis, and optionally superior to the penis base 1106. In some embodiments, the device is attached to a body region superior to the base of the penis, or to a body region at the base 1106 of the penis. In some embodiments, each of at least two electrodes of the device, for example electrodes 1118 and 1120 is located at an opposite side of the penis. In some embodiments, positioning at least two electrodes at opposite sides relative to the penis allows, for example to deliver an electric field between the at least two electrodes, and transcutanously to tissues, for example at least one nerve innervating the penis. In some embodiments, a distance between the at least two electrodes 1118 and 11120 is in a range between 1 cm-10 cm, for example 1 cm-5 cm, 2 cm-7 cm, 3 cm-10 cm or any intermediate, smaller or larger distance

According to some exemplary embodiments, for example as shown in FIGS. 11A and 11B, the body 1122 of the device 1102, is straight. Alternatively, for example as shown in FIGS. 11C and 11D a body of the device comprises an inwardly curved opening 1126, shaped to be positioned at least partly around the penis base 1106 or at least partly around the penis. Optionally, the body 1124 is shaped as an arc.

According to some exemplary embodiments, at least part of the body 1122 or 1124 is flexible, for example to conform to anatomy of a skin surface which is superior to a base of the penis. Alternatively or additionally, at least part of the body 1122 or 1124 is flexible and/or bendable, for example to conform to the movement of a subject and/or to prevent discomfort during sexual intercourse. In some embodiments, at least a portion of the body 1122 or 1124 has a minimal radius of tortuosity of about 2 mm, for example a minimal radius of tortuosity of about 2.5 mm, a minimal radius of tortuosity of about 3 mm, a minimal radius of tortuosity of about 3.5 mm, a minimal radius of tortuosity of about 4 mm, or any intermediate, smaller or larger value.

According to some exemplary embodiments, the device is configured to deliver an electric field between at least two electrodes, for example electrodes 1118 and 1120, and transcutanously to at least one nerve, for example a nerve of the autonomic nervous system innervating at least one blood vessel of the penis, for example the common penile artery, a dorsal artery of the penis, a bulbourethral artery, and/or an internal pudendal artery. Reference is now made to FIG. 12, depicting positioning of a sexual arousal device at a perineal region of a subject, according to some exemplary embodiments of the invention. According to some exemplary embodiments, for example as shown in FIG. 12, a sexual arousal device, for example a device 1130 is shaped and sized to be attached to a perineum 1132 of a male subject. In some embodiments, the device 1130 is shaped and sized to be positioned between a posterior aspect 1134 of a scrotum 1136 and an anus 1138 of the subject. In some embodiments, an axial length 1139 of the device 1130 is smaller than 7 cm, for example smaller than 6 cm, smaller than 5 cm, smaller than 3 cm or any intermediate, smaller or larger value. In some embodiments, the axial length 1139 is shorter than an anogenital distance of the subject. In some embodiments, a maximal width 1142 of the device body 1144 is in a range of 5 cm to 25 cm, for example 5 cm to 15 cm, 5 cm to 10 cm, 7 cm to 20 cm or any intermediate, smaller or larger range of values.

According to some exemplary embodiments, the body 1144 comprises at least one concave portion, for example an inwardly curved opening 1140. In some embodiments, the at least one concave portion is configured to fit an anatomy of the posterior aspect 1134 of the scrotum 1136. In some embodiments, when the device 1130 is positioned at the perineum 1132, the at least one concave portion is oriented to face the posterior aspect 1134 of the scrotum 1136.

According to some exemplary embodiments, at least a portion of the device is flexible, for example to allow the device to conform to an anatomy of the perineum.

It is expected that during the life of a patent maturing from this application many relevant electrodes will be developed; the scope of the term electrode is intended to include all such new technologies a priori.

As used herein with reference to quantity or value, the term “about” means “within +10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A sexual modulation device, comprising:
a skin patch comprising a body, wherein said skin patch has a surface configured to attach said skin patch to a skin surface of an anatomical region located near a genital organ of a subject;
wherein said skin patch body, comprises:
a central portion, at least two extensions extending from said central portion, wherein said at least two extensions comprise a first extension and a second extension;
at least one opening between said first extension and said second extension shaped and sized to receive at least part of said genital organ;
at least two electrodes, suitable to transcutaneously deliver electrical impulses, wherein each of said at least two electrodes is located at a different extension of said first extension and said second extension, and at opposite sides of said genital organ positioned within said opening;
an electronic circuitry and power supply operationally connected to the at least two electrodes;
wherein said electronic circuitry and power supply are configured to deliver said electrical impulses between said at least two electrodes; and wherein said central portion is soft enough to be used during sexual intercourse.

2. A device according to claim 1, wherein at least one of, said device body, said central portion or at least an edge of said central portion is flexible.

3. A device according to claim 1, wherein said electronic circuitry and power supply are configured to deliver said electrical impulses to a depth of at least 3 mm into the skin.

4. A device according to claim 1, wherein said central portion and/or said at least two extensions have a maximal thickness of up to 7 mm.

5. A device according to claim 1, wherein said central body and/or said at least two extensions have a minimal radius of tortuosity of about 2.5 mm.

6. A device according to claim 1, wherein hardness of said central body and/or said at least two extensions is in a range of 25-90 shore A.

7. A device according to claim 1, wherein hardness of said central body and/or said at least two extensions is in a range of 15-70 shore OO.

8. A device according to claim 1, wherein said at least two extensions are shaped as wings or petals.

9. A device according to claim 1, wherein said electronic circuitry and power supply are configured to deliver said electrical impulses with parameter values selected to increase blood volume in at least one external sex organ.

10. A device according to claim 9, wherein said genital organ comprises a Clitoris of a female subject and wherein said at least one external sex organ comprise at least one of said Clitoris, a Labia Majora, a Labia Minora, and a Vestibule of said female subject.

11. A device according to claim 10, wherein said skin patch surface is configured to attach said skin patch to a skin surface of a Mons Pubis of said female subject at a distance of up to 5 cm from said Clitoris, and wherein said at least one opening is shaped and sized to receive at least part of said Clitoris.

12. A device according to claim 10, wherein said electronic circuitry and power supply are configured to deliver said electrical impulses with parameter values selected to affect at least one nerve of an autonomic nervous system innervating said Clitoris and/or Labia, or tissues associated thereof.

13. A device according to claim 12, wherein said tissues associated thereof comprise at least one smooth muscle and/or at least one blood vessel associated with the Clitoris and/or the Labia.

14. A device according to claim 13, wherein said at least one blood vessel comprises at least one dorsal clitoral artery, at least one perineal artery, at least one deep artery of the clitoris, at least one posterior labial artery or any combination thereof.

15. A device according to claim 12, wherein said at least one nerve is a sympathetic nerve and/or a parasympathetic nerve.

16. A device according to claim 15, wherein said electronic circuitry and power supply are configured to deliver said electrical impulses with a frequency in a range of 0.5 Hz-10 Hz, selected to increase activity of said at least one sympathetic nerve and/or to reduce activity of said parasympathetic nerve.

17. A device according to claim 15, wherein said electronic circuitry and power supply are configured to deliver said electrical impulses with a frequency in a range of 50 Hz to 200 Hz, selected to reduce activity of said at least one sympathetic nerve and/or to increase activity of said at least one sympathetic nerve.

18. A device according to claim 9, wherein said genital organ comprises a base of a Penis of a male subject, and wherein said external sex organ comprises said Penis.

19. A device according to claim 18, wherein said skin patch surface is configured to attach said skin patch to a surface of a skin region superior to a base of said Penis, at a distance of up to 5 cm from said Penis base, and wherein said opening is shaped and sized to receive at least part of said Penis base.

20. A device according to claim 19, wherein said electronic circuitry and power supply are configured to deliver said electrical impulses with parameter values selected to affect at least one nerve of an autonomic nervous system innervating said penis, or tissues associated with said penis.

21. A device according to claim 20, wherein said tissue associated with said penis comprise at least one blood vessel and/or a Corpus Cavernosum of said penis.

22. A device according to claim 21, wherein said at least one blood vessel comprise at least one of, an internal pudental artery, a bulbouretheral artery, a circumfex artery, and a dorsal artery.

23. A device according to claim 20, wherein said at least one nerve is a sympathetic nerve and/or a parasympathetic nerve.

24. A device according to claim 23, wherein said electronic circuitry and power supply are configured to deliver said electrical impulses with a frequency in a range of 50 Hz-200 Hz, selected to reduce activity of said at least one sympathetic nerve and/or to increase activity of said parasympathetic nerve.

25. A device according to claim 1, wherein a width of said opening between said at least two extensions is in a range of 2 cm to 20 cm.

26. A device according to claim 1, wherein a length of said opening between said at least two extensions is in a range of 1 cm-10 cm.

27. A device according to claim 1, wherein said electronic circuitry comprises a printed circuit board connecting said power supply to said at least two electrodes.

28. A device according to claim 1, wherein said skin patch body comprises at least one sensor for sensing a presence of at least one additional device, and wherein said electronic circuitry is configured to deliver said electric field according to a presence of said at least one additional device.

29. A device according to claim 28, wherein said electronic circuitry is configured to initiate delivery of said electric field, stop the delivery of said electric field, and/or modify the delivery of the electric field according to the presence of said at least one additional device.

30. A device according to claim 28, wherein said at least one additional device comprises at least one additional sexual arousal device used by at least one additional subject, at least one sex toy, and/or a condom.

31. A device according to claim 28, wherein said at least one sensor comprises a proximity sensor or a proximity detector, and wherein said at least one sensor is configured to detect a RF tag or an electronic circuitry of said at least one additional device.

32. A device according to claim 31, wherein said at least one sensor comprises a Radio Frequency Identification (RFID) reader, and wherein said tag comprises a RFID tag.

33. A device according to claim 1, wherein said skin patch body comprises a communication circuitry, configured to receive wireless signals to a remote device, and wherein said electronic circuitry is configured to deliver and/or to stop the delivery of said electrical impulses based on signals received from said remote device.

34. A device according to claim 33, wherein said remote device comprises a remote control, a cellular device, a mobile device, a mobile computer, and/or a wearable device.

35. A device according to claim 1, wherein said skin patch body comprises a user interface operationally connected to said electronic circuitry, configured to deliver a human detectable indication to a user of the device.

36. A device according to claim 35, wherein said electronic circuitry signals said user interface to deliver said human detectable indication, when the device is in a standby mode, prior to stopping the electric field delivery, when the at least two electrodes are in contact with the skin surface and/or when at least one electrode of the at least two electrodes is not in contact with the skin surface.

37. A device according to claim 35, wherein said user interface comprises at least one vibrator configured to deliver a sensory human detectable indication to the user of the device.

38. A device according to claim 1, wherein a maximal thickness of said central portion is larger than a maximal thickness of said at least two extensions.

39. A device according to claim 1, comprises an adhesive interface configured to attach said skin patch surface to said skin surface.

40. A device according to claim 39, wherein said adhesive interface is replaceable.

41. A device according to claim 1, wherein said device is disposable.

42. A device according to claim 1, wherein said skin patch surface comprises two or more electrically conducting protrusions configured to electrically interconnect said at least two electrodes to said skin surface.

43. A method for sexual modulation in a female subject, comprising:
attaching a device including a skin patch, at least two electrodes an electronic circuitry and power supply, to a skin surface of an anatomical region located near a genital organ of a female subject, prior to initiating sexual activity;
activating said device to generate and deliver an electric field into said skin surface by said at least two electrodes prior to initiating said sexual activity, wherein said electric field is delivered with parameter values selected to increase sexual arousal of said female subject;
participating in sexual activity which includes penetrative sex and/or cunnilingus of said female subject while said device is attached to said skin surface and delivers said electric field, thereby increasing sexual arousal of said female subject during said sexual activity.

44. A method according to claim 43, wherein said attaching comprises attaching said device at a distance of up to 5 cm from a clitoris of said female subject.

45. A method according to claim 44, wherein said attaching comprises attaching said device to a mons pubis of said female subject.

46. A method according to claim 44, wherein said attaching comprises positioning each electrode of said at least two electrodes at opposite sides of said clitoris.

47. A method according to claim 43, comprising delivering a human detectable indication to said female subject following activation of said device and/or prior to stopping the delivery of said electric field.

48. A method according to claim 43, comprising communicating with at least one remote device, and wherein said activating comprises activating said sexual arousal device according to signals received from said at least one remote device.

49. A method according to claim 48, wherein said activating comprises, initiating the delivery of said electric field, stopping the delivery of said electric field and/or modifying said electric field according to signals received from said at least one remote device.

50. A method according to claim 48, wherein said at least one remote device comprises, at least one additional sexual modulation device coupled to a subject participating in said sexual activity, a sex toy, a cellular device, a mobile device, a remote control and/or a computer.

51. A method according to claim 43, wherein said sexual activity comprises pelvic-pelvic contact.

52. A method for sexual modulation in a male subject, comprising:
attaching a device including a skin patch, at least two electrodes an electronic circuitry and power supply, to a skin surface of an abdomen superior to a base of a penis of said male subject, prior to initiating sexual activity;
activating said device to generate and deliver an electric field into said skin surface by said at least two electrodes prior to initiating sexual intercourse, wherein said electric field is delivered with parameter values selected to increase penile rigidity of a penis of said male subject.

53. A method according to claim 52, comprising:
participating in sexual activity with said male subject while said device is attached to said skin surface and delivers said electric field.

54. A method according to claim 53, wherein said sexual activity comprises pelvic-pelvic contact.

55. A method according to claim 52, wherein said attaching comprises attaching said device at a distance of up to 5 cm from said penis base.

56. A method according to claim 52, wherein said attaching comprises positioning each electrode of said at least two electrodes at opposite sides of said penis base.

57. A method according to claim 52, comprising delivering a human detectable indication to said male subject following activation of said device and/or prior to stopping the delivery of said electric field.

58. A method according to claim 52, comprising communicating with at least one remote device, and wherein said activating comprises activating said device according to signals received from said at least one remote device.

59. A method according to claim 58, wherein said activating comprises, initiating the delivery of said electric field, stopping the delivery of said electric field and/or modifying said electric field according to signals received from said at least one remote device.

60. A method according to claim 58, wherein said at least one remote device comprises, at least one additional sexual modulation device coupled to a subject participating in said sexual activity, a sex toy, a cellular device, a mobile device, a remote control and/or a computer.
